(19) United States
(12) Patent Application Publication
(10) Pub. No.: US 2025/0216395 A1
(43) Pub. Date: Jul. 3, 2025

(54) COMPOSITIONS AND METHODS COMPRISING ANTI-NRP2 ANTIBODIES

(71) Applicant: aTyr Pharma, Inc., San Diego, CA (US)

(72) Inventors: Luke G. BURMAN, San Diego, CA (US); Yeeting CHONG, San Diego, CA (US); Kaitlyn RAUCH, San Diego, CA (US); Leslie A. NANGLE, San Diego, CA (US)

(21) Appl. No.: 18/875,942

(22) PCT Filed: Jun. 15, 2023
(86) PCT No.: PCT/US2023/068511

§ 371 Date: Dec. 17, 2024

Related U.S. Application Data
(60) Provisional application No. 63/353,285, filed on Jun. 17, 2022.

(51) Int. Cl. G01N 33/574; C07K 16/28

(52) U.S. Cl. CPC G01N 33/57492; C07K 16/28; C07K 2317/33; C07K 2317/92; G01N 2474/20

(57) ABSTRACT

Provided are antibodies, and antigen-binding fragments thereof, that specifically bind to select human neuropilin-2 (NRP2) isoforms with low cross-reactivity to human neuropilin-1 (NRP1) and non-human NRP2, and which are optimized for diagnostic uses such as immunohistochemical or immunofluorescence assays. Also included are related compositions and methods for detecting and measuring human NRP2 in a biological sample.

### CROSS-REFERENCE TO RELATED APPLICATIONS

This application claims the benefit under 35 U.S.C. § 119 (e) of U.S. Provisional Application No. 63/353,285, filed Jun. 17, 2022, which is incorporated by reference in its entirety.

### STATEMENT REGARDING THE SEQUENCE LISTING

The Sequence Listing XML associated with this application is provided in XML file format and is hereby incorporated by reference into the specification. The name of the XML file containing the Sequence Listing XML is ATYR_139_01WO_ST26.xml. The XML file is about 39,352 bytes, was created on Jun. 14, 2023, and is being submitted electronically via USPTO Patent Center.

### BACKGROUND

### Technical Field

The present disclosure relates to antibodies, and antigen-binding fragments thereof, that specifically bind to select human neuropilin-2 (NRP2) isoforms with low cross-reactivity to human neuropilin-1 (NRP1) and non-human NRP2, and which are optimized for diagnostic uses such as immunohistochemical or immunofluorescence assays. Also included are related compositions and methods for detecting and measuring human NRP2 in a biological sample.

### Description of the Related Art

Neuropilin-2 (NRP2) is a cell surface receptor that was originally discovered as a neuronal guidance molecule, but has more recently been linked to a broad range of cellular processes, including EMT, cell migration, lymphangiogenesis, and inflammatory conditions. In cancer, NRP2 over-expression is linked to more aggressive growth, cellular invasion, metastatic growth, and resistance development. NRP2 interacts with a broad array of signal transduction, and receptor signaling systems, and is also involved in pinocytosis and efferocytosis. Given the broad range of cellular processes regulated by NRP2, there is a need for high affinity detection antibodies which show high selectivity for human NRP2, bind to all major splice variants of human NRP2, and discriminate between NRP1 and NRP2. However, to date, the availability of reagents for immunohistochemical (IHC) detection of NRP2 protein in patient tissue samples has been less than ideal, in the least because commercially available anti-NRP2 antibodies display high non-specific staining. Accordingly, there is a need in the art for robust, high-affinity diagnostic and research reagents that can selectively detect NRP2.

The present disclosure satisfies such needs, for example, by describing the development and characterization of a highly-selective, high-affinity antibody that robustly binds to both NRP2a and NRP2b, and which shows low non-specific binding to other cellular components.

### BRIEF SUMMARY

Embodiments of the present disclosure include an isolated antibody, or an antigen-binding fragment thereof, which binds to a human neuropilin-2 (NRP2) polypeptide, and comprises a heavy chain variable region (V_{H}) sequence that comprises complementary determining region V_{H}CDR1, V_{H}CDR2, and V_{H}CDR3 sequences set forth in SEQ ID NOs: 1-3, respectively; and a light chain variable region (V_{L}) sequence that comprises complementary determining region V_{L}CDR1, V_{L}CDR2, and V_{L}CDR3 sequences, set forth in SEQ ID NOs: 4-6, respectively, including variants thereof having 1, 2, 3, 4, 5, or 6 total alterations across all of the CDR regions.

In some embodiments, the V_{H} sequence comprises an amino acid sequence that is at least 80, 85, 90, 95, 97, 98, 99, or 100% identical to SEQ ID NO: 7, and the V_{L} sequence comprises an amino acid sequence that is at least 80, 85, 90, 95, 97, 98, 99, or 100% identical to SEQ ID NO: 8. In some embodiments, the isolated antibody, or antigen-binding fragment thereof, binds to a denatured form of the human NRP2 polypeptide. In some embodiments, the isolated antibody, or antigen-binding fragment thereof, binds to each of the human NRP2 polypeptides selected from human neuropilin-2a (NRP2a) variant 1 (v1), human NRP2a variant 2 (v2), human NRP2a variant 3 (v3), human neuropilin-2b (NRP2b) variant 4 (v4), and human NRP2b variant 5 (v5), optionally wherein the NRP2 polypeptides are selected from Table N1. In some embodiments, the isolated antibody, or antigen-binding fragment thereof, binds to the human NRP2 polypeptide at an epitope that comprises SEQ ID NO: 25. In some embodiments, the isolated antibody, or antigen-binding fragment thereof, binds to the human NRP2 polypeptide with an affinity of about or less than about 10 nM. In some embodiments, the isolated antibody, or antigen-binding fragment thereof, does not substantially bind to cynomolgus NRP2, mouse NRP2, rat NRP2, or human neuropilin-1 (NRP1).

In some embodiments, the isolated antibody, or antigen-binding fragment thereof, comprises an IgA (including subclasses IgA1 and IgA2), IgD, IgE, IgG (including subclasses IgG1, IgG2, IgG3, and IgG4), or IgM Fc domain. In some embodiments, the isolated antibody, or antigen-binding fragment thereof, is a monoclonal antibody. In some embodiments, the isolated antibody, or antigen-binding fragment thereof, comprises a mouse, rabbit, or goat IgG Fc domain, optionally a IgG1 or IgG2A Fc domain.

In some embodiments, the isolated antibody, or antigen-binding fragment thereof, is covalently attached to a detectable label. In certain embodiments, the detectable label is selected from one or more of a fluorophore/fluorescent dye, polymer particle label, metal particle label, iodine-based label, alkaline phosphatase, horseradish peroxidase, luminescent label, radioactive label or radioisotope, nanoparticle, and a quantum dot.

Also included are compositions, comprising the isolated antibody, or antigen-binding fragment thereof, as described herein, and a suitable carrier. In certain embodiments, the composition of the isolated antibody, or antigen-binding fragment thereof, is lyophilized.

A method of determining an amount of a human NRP2 polypeptide in a biological sample, comprising
(a) contacting the biological sample with an antibody, or antigen-binding fragment thereof, as described herein; and
(b) determining the amount of the antibody, or antigen-binding fragment thereof, in the biological sample, which determines the amount of the human NRP2 polypeptide in the biological sample.

Also included are methods of identifying an NRP2-expressing cancer in a biological sample of cancer tissue from a subject, comprising
(a) contacting the biological sample with an antibody, or antigen-binding fragment thereof, as described herein;
(b) determining the amount or subcellular localization of the antibody, or antigen-binding fragment thereof, in the biological sample, which determines the amount or subcellular localization of NRP2 in the biological sample; and
(c) identifying the NRP2-expressing cancer if (i) the amount of NRP2 in the biological sample of cancer tissue from the subject is increased relative to a control or reference, or (ii) the subcellular localization of the NRP2 is increased relative to a control or reference.

In certain embodiments, the subcellular localization of the NRP2 is increased in the nucleus or nuclear envelope relative to the control or reference. In certain embodiments, step (b) comprises determining the ratio of NRP2 localized in the nucleus or nuclear envelope (nuclear NRP2) relative to NRP2 localized on the cell surface (cell surface NRP2), and (c) comprises identifying the NRP2-expressing cancer if the ratio of nuclear NRP2/cell surface NRP2 is increased relative to a control or reference. Some embodiments include administering or causing to be administered to the subject having the NRP2-expressing cancer of (c) at least one NRP2-targeted therapeutic agent, optionally a therapeutic antibody, or antigen-binding fragment thereof, which binds to human NRP2, and optionally in combination with at least one additional anti-cancer therapy or agent.

Some embodiments relate to methods of identifying a subject for NRP2-targeted therapy, comprising
(a) contacting a biological sample from the subject with an antibody, or antigen-binding fragment thereof, as described herein;
(b) determining the amount or subcellular localization of the antibody, or antigen-binding fragment thereof, in the biological sample, which determines the amount or subcellular localization of NRP2 in the sample; and
(c) identifying the subject as suitable for NRP2-targeted therapy if (i) the amount of the NRP2 in the biological sample is increased relative to a control or reference, or (ii) the subcellular localization of the NRP2 is increased relative to a control or reference.

In certain embodiments, the subcellular localization of the NRP2 is increased in the nucleus or nuclear envelope relative to the control or reference. In certain embodiments, step (b) comprises determining the ratio of NRP2 localized in the nucleus or nuclear envelope (nuclear NRP2) relative to NRP2 localized on the cell surface (cell surface NRP2), and (c) comprises identifying the NRP2-expressing cancer if the ratio of nuclear NRP2/cell surface NRP2 is increased relative to a control or reference. Some embodiments include administering or causing to be administered to the subject of (c) at least one NRP2-targeted therapeutic agent, optionally a therapeutic antibody, or antigen-binding fragment thereof, which binds to human NRP2.

Also included are methods for identifying a treatment regimen for a subject with castration-resistant prostate cancer (CRPC) that is relapsed/refractory to androgen receptor (AR)-targeted therapy, comprising
(a) contacting a biological sample from the subject with an antibody, or antigen-binding fragment thereof, as described herein;
(b) determining the subcellular localization of the antibody, or antigen-binding fragment thereof, in the biological sample, which determines the subcellular localization of NRP2 in the sample; and
(c) identifying the subject as being suitable for an aggressive treatment regimen if the subcellular localization of the NRP2 in the nucleus or nuclear envelope is increased relative to a control or reference.

In certain embodiments, step (b) comprises determining the ratio of NRP2 localized in the nucleus or nuclear envelope (nuclear NRP2) relative to NRP2 localized on the cell surface (cell surface NRP2), and (c) comprises identifying the subject as being suitable for the aggressive treatment regimen if the ratio of nuclear NRP2/cell surface NRP2 is increased relative to a control or reference. In some embodiments, the aggressive treatment regimen comprises at least one NRP2-targeted therapeutic agent, optionally a therapeutic antibody, or antigen-binding fragment thereof, which binds to human NRP2, in combination with at least one additional anti-cancer therapy or agent optionally at least one chemotherapeutic agent.

Some embodiments include methods of identifying NRP2-mediated drug resistance in a subject, comprising
(a) contacting a biological sample from the subject with an antibody, or antigen-binding fragment thereof, as described herein;
(b) determining the subcellular localization of the antibody, or antigen-binding fragment thereof, in the biological sample, which determines the subcellular localization of NRP2 in the sample; and
(c) identifying the subject as having NRP2-mediated drug resistance if the subcellular localization of NRP2 to the nucleus or nuclear envelope is increased relative to a control or reference.

Certain embodiments comprise administering or causing to be administered to the subject of (c) at least one NRP2-targeted therapeutic agent, optionally a therapeutic antibody, or antigen-binding fragment thereof, which binds to human NRP2, optionally in combination with at least one additional anti-cancer therapy or agent, optionally radiotherapies, cancer immunotherapies, chemotherapeutic agents (optionally DNA damaging agents, DNA repair inhibitors), hormonal therapeutic agents, kinase inhibitors, anti-growth factor therapies, and androgen receptor (AR)-targeted therapies. In certain embodiments, step (c) comprises correlating a higher increase in subcellular localization of NRP2 to the nucleus or nuclear envelope with a more advanced stage of NRP2-mediated drug resistance.

Some embodiments comprise first obtaining the biological sample from a subject, optionally by receiving the biological sample from a healthcare provider, optionally wherein the subject has or is suspected of having a cancer. Certain embodiments comprise providing information to a healthcare provider on the amount or subcellular localization of NRP2 in the biological sample. In certain embodiments, the information on the subcellular localization of NRP2 comprises a ratio of nuclear NRP2/cell surface NRP2 in the biological sample.

In certain embodiments, the biological sample is a biopsy sample. In certain embodiments, the biopsy sample is a cancer or suspected cancer biopsy sample. In some embodiments, the biopsy sample is selected from skin tissue, liver tissue, pancreatic tissue, prostate tissue, mesothelial tissue, epithelial tissue, ovarian tissue, colorectal tissue, gastric tissue, brain tissue, lung tissue, kidney tissue, bladder tissue, uterine tissue, esophageal tissue, cervical tissue, testicular tissue, breast tissue, and mesenchymal tissue such as bone tissue, cartilage tissue, fat tissue, muscle tissue, vascular tissue, blood, or hematopoietic cells/tissue, optionally a liquid biopsy.

In certain embodiments, the control or reference is a reference standard, a biological sample from a healthy subject, or a healthy biological sample from the same subject. In certain embodiments, the control is a non-cancerous biological sample from the same subject, optionally of the same tissue type.

In some embodiments, steps (a) and (b) comprise performing an immunohistochemistry (IHC) or immunofluorescence (IF) assay on the biological sample. In certain embodiments, the IHC or IF assay comprises a multiplex IHC or IF assay, comprising contacting the biological sample with at least one additional antibody, or antigen-binding fragment thereof, which specifically binds to an additional marker of interest. In particular embodiments, the additional marker of interest is selected from one or more of signal transduction pathway molecules (VEGF-C, VEGF-A, EGF, IGF, FGF, TGF-beta, VEGFR1, VEGFR2, VEGFR3, CCR7, EGFR1, EGFR2, PDGFR, TGFR1, TGFR2, TGFR3, and c-MET); EMT markers (N-cadherin, E-cadherin, OB-cadherin, ZO-1, α5β1 integrin 1, αVβ6 integrin, Syndecan-1, FSP1, Cytokeratin, α-SMA, Vimentin 1, β-Catenin, CDH1, EPCAM, claudins, cytokeratins, Snail, Slug, ZEB1, ZEB2, and Twist); lymphangiogenesis markers (lymphatic vessel endothelial hyaluronan receptor-1, or LYVE-1); fibrosis markers (collagen fibers, tenascin-C, and α-SMA); and immune activation/exhaustion markers and immune modulators (CD45RA, CD45RO, CD27, CD62L, CD95, PD-1, PD-L1, CD80, CD86, CXCR4, BLC, sCD30, MCP-2, IP-10, APRIL, SIL-2R, IL7, MIF, MIP-1b, SCF, SDF-1a, and sTNF-RI).

### BRIEF DESCRIPTION OF THE DRAWINGS

FIG. 1 shows ELISA binding of aNRP2-36v2 to human NRP2v2. Antibody aNRP2-36v2 titrates specifically to the NRP2v2 (SEQ ID NO: 14) protein. All three protein lots tested show similar signals to the NRP2v2 protein indicating similar activity across multiple purification lots.

FIG. 2 shows aNRP2-36v2 ELISA epitope mapping. Antibody aNRP2-36v2 only binds to the NRP2v2 (SEQ ID NO: 14) and NRP2bv5 (SEQ ID NO: 27) proteins, which indicates the epitope is located in NRP2 residues 642-808. aNRP2-36v2 also does not show any cross-reactivity with mouse, rat, or cynomolgus NRP2. The three different lots also show similar magnitude of signals to each other which also indicates the consistency between the different purification lots.

FIGS. 3A-3B show binding of aNRP2-36v2 to human NRP2v2 and cynomolgus NRP2×2 by BLI. (3A) aNRP2-36v2 binds to human NRP2v2 with an affinity of 9.7 nM. Dashed lines are the fitted data. (3B) aNRP2-36v2 does not bind to cynomolgus NRP2×2.

FIG. 4 shows aNRP2-36v2 binding to human NRP2 in the presence of blocking peptide.

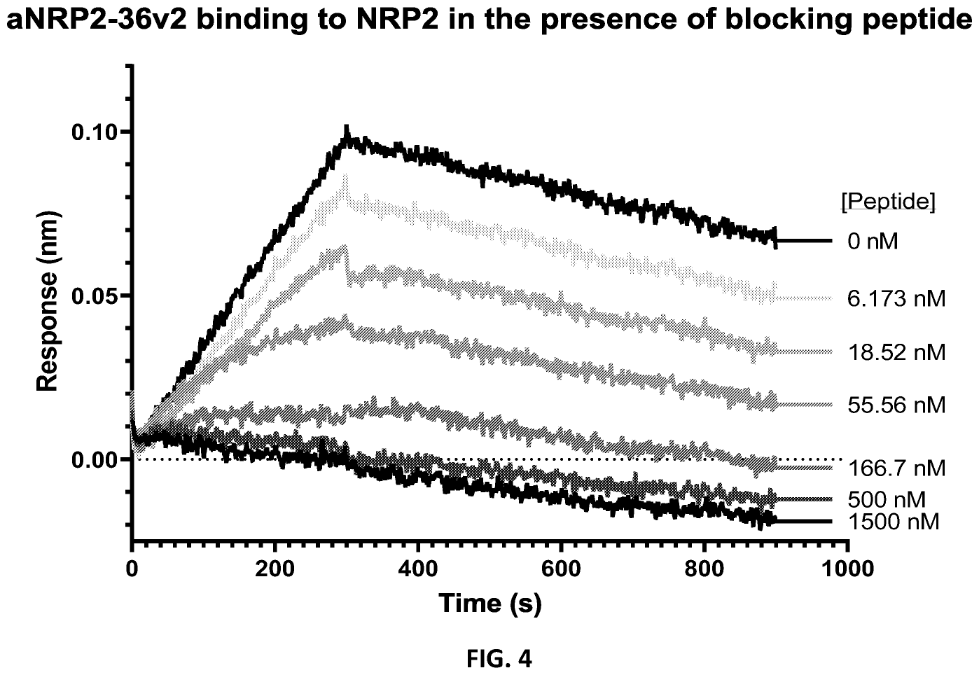

FIG. 5 shows Western blotting with aNRP2-36v2. Antibody aNRP2-36v2 only reacts to proteins NRP2v2 (23-855) and NRP2bv5 (23-832) proteins. This data is consistent with the ELISA epitope mapping data. All antibody lots consistently bind the NRP2v2 (23-855) and NRP2bv5 (23-832) proteins and do not show binding to mouse or rat NRP2, and also do not cross-react with NRP1.

FIG. 6 shows Western blot of NRP2 with aNRP2-36v2 in the presence of blocking peptide. Here, 1 μg/mL aNRP2-36v2 was incubated with either 5 μg/mL blocking peptide, 5 μg/mL control peptide, or no added peptide, and was then used in Western blotting to detect NRP2. Presence of blocking peptide prevents detection by aNRP2-36v2, indicating that the peptide is binding to the antibody and preventing its association with NRP2.

FIG. 7 shows IHC detection of NRP2 expression in sarcoidosis skin granulomas by the aNRP2-36v2 antibody.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below. All of the patent and non-patent literature references listed herein are incorporated by reference in their entireties.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight, or length.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the ε-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

The term “biological sample” includes a biological material that can be collected from a subject and used in connection with diagnosis or monitoring of biological states. Biological samples can include clinical samples, including body fluid samples, such as body cavity fluids, urinary fluids, cerebrospinal fluids, blood, and other liquid samples of biological origin; and tissue samples, such as biopsy samples, tumor or suspected tumor samples, and other solid samples of biological origin. Biological samples can also include those that are manipulated in some way after their collection, such as by treatment with reagents, culturing, solubilization, enrichment for certain biological constituents, cultures or cells derived therefrom, and the progeny thereof.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “conjugate” includes an entity formed as a result of covalent or non-covalent attachment or linkage of an agent or other molecule, e.g., a detectable label, to an antibody described herein.

A “control” such as a “control subject” or “control tissue” includes a healthy subject or a healthy tissue sample, for example, which is not pathological or diseased. In certain embodiments, a control includes a non-diseased (e.g., non-cancerous) tissue from a different, healthy subject or the same subject being tested or diagnosed. A control can also include a reference standard, for example, a standard value generated from one or more healthy subjects or tissues (e.g., a population or cohort of healthy subjects or tissues).

As used herein, the terms “function” and “functional” and the like refer to a biological, enzymatic, or therapeutic function.

“Homology” refers to the percentage number of amino acids that are identical or constitute conservative substitutions. Homology may be determined using sequence comparison programs such as GAP (Deveraux et al., Nucleic Acids Research. 12, 387-395, 1984), which is incorporated herein by reference. In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances. In particular embodiments, the isolated polypeptide is an antibody.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

In certain embodiments, the “purity” of any given agent (e.g., an antibody) in a composition may be specifically defined. For instance, certain compositions may comprise an agent that is at least 80%, 85%, 90%, 91%, 92%, 93%, 94%, 95%, 96%, 97%, 98%, 99%, or 100% pure, including all decimals in between, as measured, for example and by no means limiting, by high-performance liquid chromatography (HPLC), a well-known form of column chromatography used frequently in biochemistry and analytical chemistry to separate, identify, and quantify compounds.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations, and the like, as well as other modifications known in the art, both naturally occurring and non-naturally occurring. A polypeptide may be an entire protein, or a subsequence, fragment, variant, or derivative thereof.

The term “polynucleotide” and “nucleic acid” includes mRNA, RNA, CRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

“Expression control sequences” include regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, which have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Eukaryotic promoters can often, but not always, contain “TATA” boxes and “CAT” boxes. Prokaryotic promoters contain Shine-Dalgarno sequences in addition to the −10 and −35 consensus sequences.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically wherein the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997. A detailed discussion of sequence analysis can be found in Unit 19.3 of Ausubel et al., “Current Protocols in Molecular Biology,” John Wiley & Sons Inc., 1994-1998, Chapter 15.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

The term “solubility” refers to the property of an antibody described herein to dissolve in a liquid solvent and form a homogeneous solution. Solubility is typically expressed as a concentration, either by mass of solute per unit volume of solvent (g of solute per kg of solvent, g per dL (100 mL), mg/mL, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, or pH 7.4. In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (˜37° C.). In certain embodiments, an antibody has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg/mL at room temperature or at about 37° C.

A “subject,” as used herein, includes any animal that exhibits a symptom or condition, or is at risk for or suspected of exhibiting a symptom or condition, which can be diagnosed with an antibody described herein. Suitable subjects (patients) include laboratory animals (such as mouse, rat, rabbit, or guinea pig), farm animals, and domestic animals or pets (such as a cat or dog). Non-human primates and, preferably, human patients, are included.

A “subject subpopulation” or “patient subpopulation,” as used herein, includes a subject or patient subset characterized as having one or more distinctive measurable and/or identifiable characteristics that distinguishes the subject or patient subset from others in the broader disease category (e.g., cancer) to which it belongs. Such characteristics include disease subcategories, gender, lifestyle, health history, organs/tissues involved, treatment history, etc. In some embodiments, a patient or subject subpopulation is characterized by the (e.g., increased) amount or levels of a NRP2 polypeptide in a biological sample, for example, a tumor sample.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Substantially” or “essentially” means nearly totally or completely, for instance, 95%, 96%, 97%, 98%, 99% or greater of some given quantity.

“Substantially free” refers to the nearly complete or complete absence of a given quantity for instance, less than about 5%, 4%, 3%, 2%, 1%, 0.5% or less of some given quantity. For example, certain compositions may be “substantially free” of cell proteins, membranes, nucleic acids, endotoxins, or other contaminants.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent (e.g., anti-NRP2 antibody) needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

### Anti-NRP2 Antibodies

Embodiments of the present disclosure relate to antibodies, and an antigen-binding fragments thereof, which bind to a human neuropilin-2 (NRP2) polypeptide. NRP2 is a single transmembrane receptor with a predominant extracellular region containing two CUB domains (al/a2 combined domain), two Factor V/VIII homology domains (b1/b2 combined domain), a MAM domain (c domain), and a short juxtamembrane region that connects the c domain to the transmembrane domain (which traverses the plasma membrane). NRP2 is typically expressed in vivo as a mixture of various closely related splice variants, which are often grouped together as NRP2a, which comprises “isoforms” or “variants” v1, v2, and v3, and NRP2b, which comprises isoforms or variants v4 and v5. Variant v6 is a soluble form of NRP2 which is found in circulation.

The NRP2a and NRP2b splice variants have identical amino acid sequences over the a1, a2, b1, b2 and c domain, but differ in sequence over the juxtamembrane, transmembrane, and cytoplasmic regions. The NRP2a variants v1, v2, and v3 also differ in amino acid sequence over these regions based on their pattern of alternative splicing, with NRP2a v1 (931aa) and NRP2a v2 (926aa) having larger inserts compared to the relatively smaller NRP2a v3 (909aa). The different sizes of these alternatively spliced forms of NRP2a reflect a loss of a 5 amino acid stretch at the N-terminus of the juxtamembrane sequence from v1 to v2, then a further loss of 17 amino acids immediately C-terminal to the 5 amino acid deletion in the v3 variant. The C-terminal half of the juxtamembrane region, transmembrane helix, and cytoplasmic domain remains identical in all three NRP2a variants.

In both NRP2a and NRP2b, the a1a2 combined domain of NRP2 interacts with sema region of the semaphorins, and the b1 domain interacts with the semaphorin PSI and Ig-like domains. NRP2 has a higher affinity for SEMA3F and 3G; in contrast, SEMAs 3A, 3B and 3E preferentially interact with NRP1. Both NRP1 and NRP2 have similar affinity for SEMA 3C. The b1b2 combined domain of NRP2 interacts with several growth factors containing heparin-binding domains, including VEGF C & D, placental growth factor (PIGF)-2, fibroblast growth factor (FGF), galectin, hepatocyte growth factor (HGF), platelet derived growth factor (PDGF), and transforming growth factor (TGF)-beta (see, for example, Prud'homme et al., Oncotarget. 3:921-939, 2012). NRP2 also interacts with various growth factor-specific receptors, and interactions with these receptors can occur independently of binding to SEMAs. In this context, integrins and growth factor receptors like VEGFR2 and VEGFR3, TGF-beta receptors, c-Met, EGFR, FGFR, PDGFR, have been shown to interact with NRPs and in general appear to increase the affinity of each ligand for its receptor and to modulate downstream signaling. The c domain (Mam) domain does not appear to be directly required for ligand binding, but may impact ligand specificity, receptor signaling, and NRP2 dimerization.

Neuropilin-2 modulates a broad range of cellular functions through its roles as an essential cell surface receptor and co-receptor for a variety of ligands (see, e.g., Guo and Vander Kooi, J. Cell. Biol. 290 No 49:29120-29126, 2015). Additionally, NRP2 is a key player in the pathophysiology of many diseases (e.g., “NRP2-associated diseases”, as described herein) and interacts with a broad array of soluble ligands including semaphorin 3F, VEGF-C and D, and TGF-beta, and an array of cellular receptors and co-factors. For instance, NRP2 directly contributes to cancer stem cell maintenance, and survival leading to increased tumor initiation, survival, chemo- and radio-resistance development, and metastasis (see, e.g., and Samuel et al., PLOS ONE 6(10) e23208, 2011), Prud'homme et al., Oncotarget 3:921-939, 2012). Moreover, NRP2b isoforms are specifically implicated in supporting TGFβ-mediated progression in lung cancer (Gemmill et al., Sci Signal. 10(462), 2017: eaag0528), facilitating resistance to tyrosine kinase inhibitors in non-small cell lung cancer (Dimou et al., J Thorac Cardiovasc Surg. 162(2): 463-473, 2021), and correlating with clinical outcome in bladder cancer (Förster et al., Genes (Basel). 12(4): 550, 2021), among other roles in cancer.

In certain embodiments, an antibody, or an antigen-binding fragment thereof, is characterized by or comprises a heavy chain variable region (V_{H}) sequence that comprises complementary determining region V_{H}CDR1, V_{H}CDR2, and V_{H}CDR3 sequences, and a light chain variable region (V_{L}) sequence that comprises complementary determining region V_{L}CDR1, V_{L}CDR2, and V_{L}CDR3 sequences. Exemplary V_{H}, V_{H}CDR1, V_{H}CDR2, V_{H}CDR3, V_{L}, V_{L}CDR1, V_{L}CDR2, and V_{L}CDR3 sequences are provided in Table S1 and Table S2 below.

TABLE S1
Exemplary CDR Sequences

| | | |
|---|---|---|
| | | SEQ ID |
| Description | Sequence | NO: |

aNRP2-36v2

| | | |
|---|---|---|
| V_{H}CDR1 | GYSFTDYNMN | 1 |
| V_{H}CDR2 | VINPKSGTTVYNQKFKG | 2 |
| V_{H}CDR3 | ADSSGGC | 3 |
| V_{L}CDR1 | KSTQRLLDSDGKTYLN | 4 |
| V_{L}CDR2 | LMSKLDS | 5 |
| V_{L}CDR3 | WQGTHFPWT | 6 |

TABLE S2
Exemplary Polypeptide Sequences

| | | |
|---|---|---|
| | | SEQ |
| Descrip- | | ID |
| tion | Sequence | NO: |

aNRP2-36v2

| | | |
|---|---|---|
| Heavy | ELQLQQSGPELVKPGASVKISCKASGYSFTDYN | 7 |
| chain | MNWVKQSNGKSLEWIGVINPKSGTTVYNQKFKG | |
| variable | KATLTVDQSSSTAYMQLNSLTSEDSAVYYCARA | |
| region | DSSGGCWGQGTTLTVSS | |
| (V_{H}) | | |
| Light | DVVMTQTPLTLSVTIGQPASISCKSTQRLLDSD | 8 |
| chain | GKTYLNWLLQRPGQSPKRLIYLMSKLDSGVPDR | |
| variable | FTGSGSGTDFTLKISRVEAEDLGVYYCWQGTHF | |
| region | PWTFGGGTKLEIK | |
| (V_{L}) | | |

Thus, in certain embodiments, an antibody or antigen-binding fragment thereof comprises:
a heavy chain variable region (V_{H}) sequence that comprises complementary determining region V_{H}CDR1, V_{H}CDR2, and V_{H}CDR3 sequences selected from Table S1 and variants thereof which specifically bind to a human NRP2 polypeptide or an epitope thereof (selected, for example, from Table N1); and
a light chain variable region (V_{L}) sequence that comprises complementary determining region V_{L}CDR1, V_{L}CDR2, and V_{L}CDR3 sequences selected from Table S1 and variants thereof which specifically bind to the human NRP2 polypeptide or an epitope thereof (selected, for example, from Table N1).

In certain embodiments, the V_{H}CDR1, V_{H}CDR2, and V_{H}CDR3 sequences comprise SEQ ID NOs: 1-3, respectively, and the V_{L}CDR1, V_{L}CDR2, and V_{L}CDR3 sequences comprise SEQ ID NOs: 4-6, respectively. Also included are variants thereof, including affinity matured variants, which bind to a human NRP2 polypeptide or epitope thereof (see, for example, Table N1), for example, variants having 1, 2, 3, 4, 5, or 6 total alterations across all of the CDR regions, for example, one or more the V_{H}CDR1, V_{H}CDR2, V_{H}CDR3, V_{L}CDR1, V_{L}CDR2, and/or V_{L}CDR3 sequences described herein. Exemplary “alterations” include amino acid substitutions, additions, and deletions.

In certain embodiments, the V_{H} sequence comprises a sequence that is at least 80, 85, 90, 95, 97, 98, 99, or 100% identical to a sequence selected from Table S2, including, for example, wherein the V_{H} sequence has 1, 2, 3, 4, 5, or 6 total alterations in one or more framework regions. In some embodiments, the V_{L} sequence comprises a sequence that is at least 80, 85, 90, 95, 97, 98, 99, or 100% identical to a sequence selected from Table S2, including, for example, wherein the V_{L} sequence has 1, 2, 3, 4, 5, or 6 total alterations in one or more framework regions.

In some embodiments, the V_{H} sequence is at least 80, 85, 90, 95, 97, 98, 99, or 100% identical to SEQ ID NO: 7, and the V_{L} sequence is at least 80, 85, 90, 95, 97, 98, 99, or 100% identical to SEQ ID NO: 8. Certain variants have 1, 2, 3, 4, 5, or 6 total alterations in one or more framework regions, which bind to a human NRP2 polypeptide or epitope thereof (see, for example, Table N1). As above, exemplary “alterations” include amino acid substitutions, additions, and deletions.

As used herein, the term “antibody” encompasses not only intact polyclonal or monoclonal antibodies, but also fragments thereof (such as dAb, Fab, Fab′, F(ab′)2, Fv), single chain (scFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. Certain features and characteristics of antibodies (and antigen-binding fragments thereof) are described in greater detail herein.

An antibody or antigen-binding fragment can be of essentially any type. As is well known in the art, an antibody is an immunoglobulin molecule capable of specific binding to a target, such as an immune checkpoint molecule, through at least one epitope recognition site, located in the variable region of the immunoglobulin molecule.

The term “antigen-binding fragment” as used herein refers to a polypeptide fragment that contains at least one CDR of an immunoglobulin heavy and/or light chain that binds to the antigen of interest. In this regard, an antigen-binding fragment of the herein described antibodies may comprise 1, 2, 3, 4, 5, or all 6 CDRs of a V_{H} and V_{L} sequence from antibodies that bind to a target molecule.

The binding properties of antibodies and antigen-binding fragments thereof can be quantified using methods well known in the art (see Davies et al., Annual Rev. Biochem. 59:439-473, 1990). In some embodiments, an antibody or antigen-binding fragment thereof specifically binds to a target molecule, for example, an NRP2 polypeptide or an epitope or complex thereof, with an equilibrium dissociation constant that is about or ranges from about ≤10⁻⁷ M to about 10⁻⁸ M. In some embodiments, the equilibrium dissociation constant is about or ranges from about ≤10⁻⁹ M to about ≤10⁻¹⁰ M. In certain illustrative embodiments, an antibody or antigen-binding fragment thereof has an affinity (Kd or EC₅₀) for a target molecule (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

A molecule such as a polypeptide or antibody is said to exhibit “specific binding” or “preferential binding” if it reacts or associates more frequently, more rapidly, with greater duration and/or with greater affinity with a particular cell, substance, or particular epitope than it does with alternative cells or substances, or epitopes. An antibody “specifically binds” or “preferentially binds” to a target molecule or epitope if it binds with greater affinity, avidity, more readily, and/or with greater duration than it binds to other substances or epitopes, for example, by a statistically significant amount. Typically one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. The term is also applicable where, for example, an antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. As used herein, the term “affinity” includes the equilibrium constant for the reversible binding of two agents and is expressed as Kd or EC₅₀. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution.

As noted above, in certain embodiments, an antibody, or an antigen-binding fragment thereof, binds or specifically binds to a human NRP2 polypeptide. Examples of NRP2 polypeptides include a human neuropilin-2a (NRP2a) variant 1 (v1) polypeptide, a human NRP2a variant 2 (v2) polypeptide, a human NRP2a variant 3 (v3) polypeptide, a human neuropilin-2b (NRP2b) variant 4 (v4) polypeptide, and a human NRP2b variant 5 (v5) polypeptide. The primary amino acid sequences of exemplary human and non-human NRP2 polypeptides are provided in Table N1 below.

TABLE N1

| | | SEQ ID |
|---|---|---|
| Description | Sequence | NO: |
| NRP2a | QPDPPCGGRLNSKDAGYITSPGYPQDYPSHQNCEWIVYAPEPNQKIVLNFNP | 9 |
| isoform 1 | HFEIEKHDCKYDFIEIRDGDSESADLLGKHCGNIAPPTIISSGSMLYIKFTS | |
| | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPSGFNC | |
| | NFDFLEEPCGWMYDHAKWLRTTWASSSSPNDRTFPDDRNFLRLQSDSQREGQ | |
| | YARLISPPVHLPRSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFAGENFKVDIPEIHEREGYEDEIDDEYEVDWSNSSSATSGSGAPSTDK | |
| | EKSWLYTLDPILITIIAMSSLGVLLGATCAGLLLYCTCSYSGLSSRSCTTLE | |
| | NYNFELYDGLKHKVKMNHQKCCSEA | |
| NRP2a | QPDPPCGGRLNSKDAGYITSPGYPQDYPSHQNCEWIVYAPEPNQKIVINFNP | 10 |
| isoform 2 | HFEIEKHDCKYDFIEIRDGDSESADLLGKHCGNIAPPTIISSGSMLYIKFTS | |
| | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPSGFNC | |
| | NFDFLEEPCGWMYDHAKWLRTTWASSSSPNDRTFPDDRNFLRLQSDSQREGQ | |
| | YARLISPPVHLPRSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFAVDIPEIHEREGYEDEIDDEYEVDWSNSSSATSGSGAPSTDKEKSWL | |
| | YTLDPILITIIAMSSLGVLLGATCAGLLLYCTCSYSGLSSRSCTTLENYNFE | |
| | LYDGLKHKVKMNHQKCCSEA | |
| NRP2a | QPDPPCGGRLNSKDAGYITSPGYPQDYPSHQNCEWIVYAPEPNQKIVLNFNP | 11 |
| isoform 3 | HFEIEKHDCKYDFIEIRDGDSESADLLGKHCGNIAPPTIISSGSMLYIKFTS | |
| | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPSGFNC | |
| | NFDFLEEPCGWMYDHAKWLRTTWASSSSPNDRTFPDDRNFLRLQSDSQREGQ | |
| | YARLISPPVHLPRSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFADEYEVDWSNSSSATSGSGAPSTDKEKSWLYTLDPILITIIAMSSLG | |
| | VLLGATCAGLLLYCTCSYSGLSSRSCTTLENYNFELYDGLKHKVKMNHQKCC | |
| | SEA | |
| NRP2b | QPDPPCGGRLNSKDAGYITSPGYPQDYPSHQNCEWIVYAPEPNQKIVLNFNP | 12 |
| isoform 4 | HFEIEKHDCKYDFIEIRDGDSESADLLGKHCGNIAPPTIISSGSMLYIKFTS | |
| | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPSGFNC | |
| | NFDFLEEPCGWMYDHAKWLRTTWASSSSPNDRTFPDDRNFLRLQSDSQREGQ | |
| | YARLISPPVHLPRSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFAGENFKGGTLLPGTEPTVDTVPMQPIPAYWYYVMAAGGAVLVLVSVA | |
| | LALVLHYHRFRYAAKKTDHSITYKTSHYTNGAPLAVEPTLTIKLEQDRGSHC | |
| NRP2b | QPDPPCGGRLNSKDAGYITSPGYPQDYPSHQNCEWIVYAPEPNQKIVLNFNP | 13 |
| isoform 5 | HFEIEKHDCKYDFIEIRDGDSESADLLGKHCGNIAPPTIISSGSMLYIKFTS | |
| | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPSGFNC | |
| | NFDFLEEPCGWMYDHAKWLRTTWASSSSPNDRTFPDDRNFLRLQSDSQREGQ | |
| | YARLISPPVHLPRSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFAGGTLLPGTEPTVDTVPMQPIPAYWYYVMAAGGAVLVLVSVALALVL | |
| | HYHRFRYAAKKTDHSITYKTSHYTNGAPLAVEPTLTIKLEQDRGSHC | |
| NRP2v2 (23- | QPDPPCGGRLNSKDAGYITSPGYPQDYPSHQNCEWIVYAPEPNQKIVLNFNP | 14 |
| 855) | HFEIEKHDCKYDFIEIRDGDSESADLLGKHCGNIAPPTIISSGSMLYIKFTS | |
| | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPSGFNC | |
| | NFDFLEEPCGWMYDHAKWLRTTWASSSSPNDRTFPDDRNFLRLQSDSQREGQ | |
| | YARLISPPVHLPRSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFAVDIPEIHEREGYEDEIDDEYEVDWSNSSSATSGSGAPSTDKEKSWL | |
| | Y | |
| NRP2- | QPDPPCGGRLNSKDAGYITSPGYPQDYPSHQNCEWIVYAPEPNQKIVLNFNP | 15 |
| ala2b1b2 (23- | HFEIEKHDCKYDFIEIRDGDSESADLLGKHCGNIAPPTIISSGSMLYIKFTS | |
| 595) | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | T | |
| NRP2- | GSEDCSKNFTSPNGTIESPGFPEKYPHNLDCTFTILAKPKMEIILQFLIFDL | 16 |
| a2b1b2 (145- | EHDPLQVGEGDCKYDWLDIWDGIPHVGPLIGKYCGTKTPSELRSSTGILSLT | |
| 595) | FHTDMAVAKDGFSARYYLVHQEPLENFQCNVPLGMESGRIANEQISASSTYS | |
| | DGRWTPQQSRLHGDDNGWTPNLDSNKEYLQVDLRFLTMLTAIATQGAISRET | |
| | QNGYYVKSYKLEVSTNGEDWMVYRHGKNHKVFQANNDATEVVLNKLHAPLLT | |
| | RFVRIRPQTWHSGIALRLELFGCRVTDAPCSNMLGMLSGLIADSQISASSTQ | |
| | EYLWSPSAARLVSSRSGWFPRIPQAQPGEEWLQVDLGTPKTVKGVIIQGARG | |
| | GDSITAVEARAFVRKFKVSYSLNGKDWEYIQDPRTQQPKLFEGNMHYDTPDI | |
| | RRFDPIPAQYVRVYPERWSPAGIGMRLEVLGCDWT | |
| NRP2- | QCNVPLGMESGRIANEQISASSTYSDGRWTPQQSRLHGDDNGWTPNLDSNKE | 17 |
| b1b2 (276- | YLQVDLRFLTMLTAIATQGAISRETQNGYYVKSYKLEVSTNGEDWMVYRHGK | |
| 595) | NHKVFQANNDATEVVLNKLHAPLLTRFVRIRPQTWHSGIALRLELFGCRVTD | |
| | APCSNMLGMLSGLIADSQISASSTQEYLWSPSAARLVSSRSGWFPRIPQAQP | |
| | GEEWLQVDLGTPKTVKGVIIQGARGGDSITAVEARAFVRKFKVSYSLNGKDW | |
| | EYIQDPRTQQPKLFEGNMHYDTPDIRRFDPIPAQYVRVYPERWSPAGIGMRL | |
| | EVLGCDWT | |
| NRP2-b1 (276- | QCNVPLGMESGRIANEQISASSTYSDGRWTPQQSRLHGDDNGWTPNLDSNKE | 18 |
| 430) | YLQVDLRFLTMLTAIATQGAISRETQNGYYVKSYKLEVSTNGEDWMVYRHGK | |
| | NHKVFQANNDATEVVLNKLHAPLLTRFVRIRPQTWHSGIALRLELFGCRVT | |
| NRP2 (23-642) | QPDPPCGGRLNSKDAGYITSPGYPQDYPSHQNCEWIVYAPEPNQKIVLNFNP | 19 |
| | HFEIEKHDCKYDFIEIRDGDSESADLLGKHCGNIAPPTIISSGSMLYIKFTS | |
| | DYARQGAGESLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPS | |
| NRP2 (23-611) | QPDPPCGGRLNSKDAGYITSPGYPQDYPSHQNCEWIVYAPEPNQKIVLNFNP | 20 |
| | HFEIEKHDCKYDFIEIRDGDSESADLLGKHCGNIAPPTIISSGSMLYIKFTS | |
| | DYARQGAGESLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSE | |
| mNRP2v2 (23- | QQDPPCGGRLNSKDAGYITSPGYPQDYPSHQNCEWIVYAPEPNQKIVLNFNP | 21 |
| 855) | HFEIEKHDCKYDFIEIRDGDSESADLLGKHCGNIAPPTIISSGSVLYIKFTS | |
| | DYARQGAGFSLRYEIFKTGSEDCSKNFTSPNGTIESPGFPEKYPHNLDCTFT | |
| | ILAKPRMEIILQFLTFDLEHDPLQVGEGDCKYDWLDIWDGIPHVGPLIGKYC | |
| | GTKTPSKLRSSTGILSLTFHTDMAVAKDGFSARYYLIHQEPPENFQCNVPLG | |
| | MESGRIANEQISASSTFSDGRWTPQQSRLHGDDNGWTPNLDSNKEYLQVDLR | |
| | FLTMLTAIATQGAISRETQKGYYVKSYKLEVSTNGEDWMVYRHGKNHKIFQA | |
| | NNDATEVVLNKLHMPLLTRFIRIRPQTWHLGIALRLELFGCRVTDAPCSNML | |
| | GMLSGLIADTQISASSTREYLWSPSAARLVSSRSGWFPRNPQAQPGEEWLQV | |
| | DLGTPKTVKGVIIQGARGGDSITAVEARAFVRKFKVSYSLNGKDWEYIQDPR | |
| | TQQTKLFEGNMHYDTPDIRRFDPVPAQYVRVYPERWSPAGIGMRLEVLGCDW | |
| | TDSKPTVETLGPTVKSEETTTPYPMDEDATECGENCSFEDDKDLQLPSGFNC | |
| | NFDFPEETCGWVYDHAKWLRSTWISSANPNDRTFPDDKNFLKLQSDGRREGQ | |
| | YGRLISPPVHLPRSPVCMEFQYQAMGGHGVALQVVREASQESKLLWVIREDQ | |
| | GSEWKHGRIILPSYDMEYQIVFEGVIGKGRSGEISIDDIRISTDVPLENCME | |
| | PISAFAVDIPETHGGEGYEDEIDDEYEGDWSNSSSSTSGAGDPSSGKEKSWL | |
| | Y | |
| rNRP2x2 (23- | QQDPPCGGRLNSKDAGYITSPGYPQDYPSHQNCEWVVYAPEPNQKIVLNFNP | 22 |
| 854) | HFEIEKHDCKYDFIEIRDGDSESADLLGKHCGNIAPPTIISSGSVLYIKFTS | |
| | DYARQGAGFSLRYEIFKTGSEDCSKNFTSPNGTIESPGFPEKYPHNLDCTFT | |
| | ILAKPRMEIILQFLTFDLEHDPLQVGEGDCKYDWLDIWDGIPHVGPLIGKYC | |
| | GTKTPSKLRSSTGILSLTFHTDMAVAKDGFSARYYLVHQEPPENFQCNAPLG | |
| | MESGRIANEQISASSTFSDGRWTPQQSRLHGDDNGWTPNVDSNKEYLQVDLR | |
| | FLTMLTAIATQGAISRETQKGYYVKSYKLEVSTNGEDWMVYRHGKNHKVFQA | |
| | NNDATELVLNKLHTPLLTRFIRIRPQTWHLGIALRLELFGCRVTDAPCSNML | |
| | GMLSGLIADTQISASSTREYLWSPSAARLVSSRSGWFPRNPQAQPGEEWLQV | |
| | DLGTPKTVKGVIIQGARGGDSITAMEARAFVRKFKVSYSLNGKDWEYIQDPR | |
| | TQQPKLFEGNMHYDTPDIRRFEPVPAQYVRVYPERWSPAGIGMRLEVLGCDW | |
| | TDSKPTVETLGPTVKSEETTTPYPMDEDATECGENCSFEDDKDLQLPSGFNC | |
| | NFDFPEETCGWMYDRAKWLQSTWISSANPNDRTFPDDKNFLKLQSDGGREGQ | |
| | FGRLISPPVHLPRSPVCMEFQYQAMGGHGVALQVVREARQESKLLWVIREDQ | |
| | GSEWKHGRIILPSYDMEYQIVFEGVIGKGRSGEISIDDIRISTDVPLENCME | |
| | PISAFAVDIPEIHGGEGYEDEIDDDYEGDWNNSSSTSGAGSPSSGKEKSWLY | |
| CNRP2x2 (23- | QPDPPCGGRLNSKDAGYITSPGYPQDYPSHQNCEWIVYAPEPNQKIVLNFNP | 23 |
| 854) | HFEIEKHDCKYDFIEIRDGDSESADLLGKHCGNIAPPTIISSGSMLYIKFTS | |
| | DYARQGAGESLRYEIFKTGSEDCSKNFTSPNGTIESPGFPEKYPHNLDCTFT | |
| | ILAKPKMEIVLQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTHEYLWSPSAARLVSSRAGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPVPAQYVRVYPERWSPAGIGMRLEVLGCDW | |
| | TDSKPTVETLGPTVKSEETTTPYPTDEEATECGENCSFEDDKDLQLPSGFNC | |
| | NFDFPEEPCGWMYDHAKWLRTTWASSSSPNDRTFPDDRNFLRLQSDSRREGQ | |
| | YARLISPPVHLPQSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFAVDIPEIHEREGYEDEIDDEYEVDWSNSSATSGSGAPSTDKEKSWLY | |
| NRP1v2 (22- | FRNDKCGDTIKIESPGYLTSPGYPHSYHPSEKCEWLIQAPDPYQRIMINFNP | 24 |
| 602) | HFDLEDRDCKYDYVEVFDGENENGHFRGKFCGKIAPPPVVSSGPFLFIKFVS | |
| | DYETHGAGFSIRYEIFKRGPECSQNYTTPSGVIKSPGFPEKYPNSLECTYIV | |
| | FAPKMSEIILEFESFDLEPDSNPPGGMFCRYDRLEIWDGFPDVGPHIGRYCG | |
| | QKTPGRIRSSSGILSMVFYTDSAIAKEGFSANYSVLQSSVSEDFKCMEALGM | |
| | ESGEIHSDQITASSQYSTNWSAERSRLNYPENGWTPGEDSYREWIQVDLGLL | |
| | RFVTAVGTQGAISKETKKKYYVKTYKIDVSSNGEDWITIKEGNKPVLFQGNT | |
| | NPTDVVVAVFPKPLITRFVRIKPATWETGISMRFEVYGCKITDYPCSGMLGM | |
| | VSGLISDSQITSSNQGDRNWMPENIRLVTSRSGWALPPAPHSYINEWLQIDL | |
| | GEEKIVRGIIIQGGKHRENKVFMRKFKIGYSNNGSDWKMIMDDSKRKAKSFE | |
| | GNNNYDTPELRTFPALSTRFIRIYPERATHGGLGLRMELLGCEVEAPTAGPT | |
| | TPNGNLVDE | |
| NRP2 (642- | SGFNCNEDFLEEPCGWMY | 25 |
| 659) | | |
| NRP2v2 (809- | VDIPEIHEREGY | 26 |
| 820) | | |
| NRP2v5 (23- | QPDPPCGGRLNSKDAGYITSPGYPQDYPSHQNCEWIVYAPEPNQKIVLNFNP | 27 |
| 832) | HFEIEKHDCKYDFIEIRDGDSESADLLGKHCGNIAPPTIISSGSMLYIKFTS | |
| | DYARQGAGFSLRYEIFKTGSEDCSKNFTSPNGTIESPGFPEKYPHNLDCTFT | |
| | ILAKPKMEIILQFLIFDLEHDPLQVGEGDCKYDWLDIWDGIPHVGPLIGKYC | |
| | GTKTPSELRSSTGILSLTFHTDMAVAKDGFSARYYLVHQEPLENFQCNVPLG | |
| | MESGRIANEQISASSTYSDGRWTPQQSRLHGDDNGWTPNLDSNKEYLQVDLR | |
| | FLTMLTAIATQGAISRETQNGYYVKSYKLEVSTNGEDWMVYRHGKNHKVFQA | |
| | NNDATEVVLNKLHAPLLTRFVRIRPQTWHSGIALRLELFGCRVTDAPCSNML | |
| | GMLSGLIADSQISASSTQEYLWSPSAARLVSSRSGWFPRIPQAQPGEEWLQV | |
| | DLGTPKTVKGVIIQGARGGDSITAVEARAFVRKFKVSYSLNGKDWEYIQDPR | |
| | TQQPKLFEGNMHYDTPDIRRFDPIPAQYVRVYPERWSPAGIGMRLEVLGCDW | |
| | TDSKPTVETLGPTVKSEETTTPYPTEEEATECGENCSFEDDKDLQLPSGFNC | |
| | NFDFLEEPCGWMYDHAKWLRTTWASSSSPNDRTFPDDRNFLRLQSDSQREGQ | |
| | YARLISPPVHLPRSPVCMEFQYQATGGRGVALQVVREASQESKLLWVIREDQ | |
| | GGEWKHGRIILPSYDMEYQIVFEGVIGKGRSGEIAIDDIRISTDVPLENCME | |
| | PISAFAGGTLLPGTEPTVDTVPMQPIPAYW | |

Thus, in certain embodiments, an antibody, or an antigen-binding fragment thereof, binds or specifically binds to a human NRP2 polypeptide selected from Table N1, or a fragment or epitope thereof. For instance, in some embodiments, an antibody, or an antigen-binding fragment thereof, binds or specifically binds a contiguous fragment of about or at least about 5, 6, 7, 8, 9, 10, 11, 12, 13, 14, 15, 16, 17, 18, 19, 20, 21, 22, 23, 24, 25, 26, 27, 28, 29, 30, 40, or 50 or more amino acids of a human NRP2 polypeptide selected from Table N1. In some embodiments, the antibody, or antigen-binding fragment thereof, binds to a denatured form of the human NRP2 polypeptide. In specific embodiments, the antibody, or antigen-binding fragment thereof, binds to the human NRP2 polypeptide at an epitope that comprises, consists, or consists essentially of residues 642-659 of human NRP2 (SEQ ID NO: 25).

In particular embodiments, the antibody, or antigen-binding fragment thereof, binds to the human NRP2 polypeptide with an affinity of about or less than about 10 nM. In some embodiments, the antibody, or antigen-binding fragment thereof, does not substantially bind to cynomolgus NRP2, mouse NRP2, rat NRP2, or human neuropilin-1 (NRP1). For instance, in certain embodiments, the antibody, or antigen-binding fragment thereof, binds selectively to a human NRP2 polypeptide relative to a corresponding cynomolgus NRP2, mouse NRP2, rat NRP2, or human NRP1 polypeptide, for instance, wherein its affinity for a human NRP2 polypeptide is significantly stronger than its affinity for a corresponding cynomolgus NRP2, mouse NRP2, rat NRP2, or human NRP1 polypeptide, for example, by about or at least about 2, 5, 10, 20, 30, 40, 50, 100, 500, or 1000-fold or more.

Merely for illustrative purposes, the binding interactions between an antibody, or antigen-binding fragment thereof, and an NRP2 polypeptide can be detected and quantified using a variety of routine methods, including octet and Biacore assays (for example, with appropriately tagged soluble reagents, bound to a sensor chip), FACS analyses with cells expressing a NRP2 polypeptide on the cell surface (either native, or recombinant), immunoassays, fluorescence staining assays, ELISA assays, and microcalorimetry approaches such as ITC (Isothermal Titration calorimetry). See also the Examples.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541).

Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

Also include are “monoclonal” antibodies, which refer to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

In some embodiments, an antibody, or an antigen-binding fragment thereof, described herein comprises the light chain constant regions (e.g., CL domain, kappa chain, lambda chain) or heavy chain constant regions (e.g., Fc regions) of any variety of immunoglobulin subtypes (e.g., IgA, IgD, IgE, IgG, IgM, including subclasses and combinations thereof, e.g., IgG1, IgG2, IgG3, IgG4), from any variety of mammals such as mouse, rabbit, or goat. The “Fc region” sequence is usually derived from the heavy chain of an immunoglobulin (Ig) molecule. A typical Ig molecule is composed of two heavy chains and two light chains. The heavy chains can be divided into at least three functional regions: the Fd region, the Fc region (fragment crystallizable region), and the hinge region, the latter being found only in IgG, IgA, and IgD immunoglobulins. The Fd region comprises the variable (V_{H}) and constant (CH1) domains of the heavy chains, and together with the variable (V_{L}) and constant (CL) domains of the light chains forms the antigen-binding fragment or Fab region.

In some embodiments, the Fc region of IgG, IgA, and IgD immunoglobulins comprises the heavy chain constant domains 2 and 3, designated respectively as CH2 and CH3 regions; and the Fc region of IgE and IgM immunoglobulins comprises the heavy chain constant domains 2, 3, and 4, designated respectively as CH2, CH3, and CH4 regions. The Fc region is mainly responsible for the immunoglobulin effector functions, which include, for example, complement fixation and binding to cognate Fc receptors of effector cells.

The hinge region (found in IgG, IgA, and IgD) acts as a flexible spacer that allows the Fab portion to move freely in space relative to the Fc region. In contrast to the constant regions, the hinge regions are structurally diverse, varying in both sequence and length among immunoglobulin classes and subclasses (see supra). The hinge region may also contain one or more glycosylation site(s), which include a number of structurally distinct types of sites for carbohydrate attachment. For example, IgA1 contains five glycosylation sites within a 17 amino acid segment of the hinge region, conferring significant resistance of the hinge region polypeptide to intestinal proteases. Residues in the hinge proximal region of the CH2 domain can also influence the specificity of the interaction between an immunoglobulin and its respective Fc receptor(s) (see, e.g., Shin et al., Intern. Rev. Immunol. 10:177-186, 1993).

The term “Fc region” or “Fc fragment” or “Fc” as used herein, thus refers to a portion of an antibody, or antigen-binding fragment thereof, which contains one or more of a CH2 region, a CH3 region, and/or a CH4 region from one or more selected immunoglobulin(s), including fragments and variants and combinations thereof. An “Fc region” may also include one or more hinge region(s) of the heavy chain constant region of an immunoglobulin.

In some embodiments, the Fc region comprises the CH2 region, CH3 region, CH4 region, and/or hinge region(s) of any one or more immunoglobulin classes, including but not limited to IgA, IgD, IgE, IgG, IgM, including subclasses and combinations thereof. In some embodiments, the Fc region is from an IgA immunoglobulin (e.g., mouse, rabbit, goat), including subclasses IgA1 and/or IgA2. In certain embodiments, the Fc region is from an IgD immunoglobulin (e.g., mouse, rabbit, goat). In particular embodiments, the Fc region is from an IgE immunoglobulin (e.g., mouse, rabbit, goat). In some embodiments, the Fc region is from an IgG immunoglobulin (e.g., mouse, rabbit, goat), including subclasses IgG1, IgG2, IgG3, and/or IgG4. In certain embodiments, the Fc region is from an IgM immunoglobulin (e.g., mouse, rabbit, goat).

Also included are antibodies, or antigen-binding fragments thereof, which comprise “variants” of the sequences in Table S1 or Table S2. A “variant” sequence, as the term is used herein, refers to a polypeptide or polynucleotide sequence that differs from a reference sequence disclosed herein (e.g., Table S1, Table S2, SEQ ID NOS: 1-8, by one or more substitutions, deletions (e.g., truncations), additions, and/or insertions. Certain variants thus include fragments of a reference sequence described herein. Variant polypeptides are biologically active, that is, they continue to possess the binding activity of a reference polypeptide. Such variants may result from, for example, genetic polymorphism and/or from human manipulation.

In many instances, a biologically active variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility.

In making such changes, the hydropathic index of amino acids may be considered. The importance of the hydropathic amino acid index in conferring interactive biologic function on a protein is generally understood in the art (Kyte & Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte & Doolittle, 1982). These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (±3.0); lysine (±3.0); aspartate (±3.0±1); glutamate (±3.0±1); serine (±0.3); asparagine (±0.2); glutamine (±0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within +2 is preferred, those within +1 are particularly preferred, and those within +0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) a1a, pro, gly, glu, asp, gln, asn, ser, thr; (2) cys, ser, tyr, thr; (3) val, ile, leu, met, a1a, phe; (4) lys, arg, his; and (5) phe, tyr, trp, his.

A variant may also, or alternatively, contain non-conservative changes. In a preferred embodiment, variant polypeptides differ from a native or reference sequence by substitution, deletion or addition of about or fewer than about 10, 9, 8, 7, 6, 5, 4, 3, 2 amino acids, or even 1 amino acid. Variants may also (or alternatively) be modified by, for example, the deletion or addition of amino acids that have minimal influence on the immunogenicity, secondary structure, enzymatic activity, and/or hydropathic nature of the polypeptide.

In general, variants will display at least about 30%, 40%, 50%, 55%, 60%, 65%, 70%, 75%, 80%, 85%, 90%, 91%, 92%, 93%, 94%, 95%, 96%, 97%, 98%, 99% similarity or sequence identity or sequence homology to a reference polypeptide sequence (e.g., Table S1, Table S2, SEQ ID NOs: 1-8). Moreover, sequences differing from the reference sequences by the addition (e.g., C-terminal addition, N-terminal addition, both), deletion, truncation, insertion, or substitution (e.g., conservative substitution) of about 1, 2, 3, 4, 5, 6, 7, 8, 9, or 10 amino acids (including all integers and ranges in between) but which retain the properties or activities of a parent or reference polypeptide sequence are contemplated.

In some embodiments, variant polypeptides differ from reference sequence by at least one but by less than 10, 9, 8, 7, 6, 5, 4, 3 or 2 amino acid residue(s). In other embodiments, variant polypeptides differ from a reference sequence by at least 1% but less than 10% or 5% of the residues. (If this comparison requires alignment, the sequences should be aligned for maximum similarity. In some instances, “looped” out sequences from deletions or insertions, or mismatches, are considered differences.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (J. Mol. Biol. 48:444-453, 1970) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6. A preferred set of parameters includes a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (Cabios. 4:11-17, 1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215:403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (Nucleic Acids Res. 25:3389-3402, 1997). When utilizing BLAST and Gapped BLAST programs, the default parameters of the respective programs (e.g., XBLAST and NBLAST) can be used.

In some embodiments, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e⁻¹¹⁷ means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In one embodiment, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mol Biol. 48:443-453, 1970) to find the alignment of two complete sequences that maximizes the number of matches and minimizes the number of gaps.

In one particular embodiment, the variant polypeptide comprises an amino acid sequence that can be optimally aligned with a reference polypeptide sequence (see, e.g., Table S1, Table S2, SEQ ID NOs: 1-8) to generate a BLAST bit scores or sequence similarity scores of at least about 50, 60, 70, 80, 90, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more, including all integers and ranges in between, wherein the BLAST alignment used the BLOSUM62 matrix, a gap existence penalty of 11, and a gap extension penalty of 1.

As noted above, a reference polypeptide may be altered in various ways including amino acid substitutions, deletions, truncations, additions, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (PNAS USA. 82:488-492, 1985); Kunkel et al., (Methods in Enzymol. 154:367-382, 1987), U.S. Pat. No. 4,873,192, Watson, J. D. et al., (“Molecular Biology of the Gene,” Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by such modifications, and for screening cDNA libraries for gene products having a selected property are known in the art. Such methods are adaptable for rapid screening of the gene libraries generated by combinatorial mutagenesis of reference polypeptides. As one example, recursive ensemble mutagenesis (REM), a technique which enhances the frequency of functional mutants in the libraries, can be used in combination with the screening assays to identify polypeptide variants (Arkin and Yourvan, PNAS USA 89:7811-7815, 1992; Delgrave et al., Protein Engineering. 6:327-331, 1993).

In certain embodiments, the antibody, or antigen-binding fragment thereof, is conjugated or covalently attached to a detectable label, for example, to facilitate detection. Exemplary detectable labels include, without limitation fluorophore/fluorescent dyes, polymer particle labels, metal particle labels, iodine-based labels, alkaline phosphatase, horseradish peroxidase, luminescent labels, radioactive labels or radioisotopes, nanoparticles, and quantum dots.

Examples of fluorophores or fluorochromes or fluorescent dyes that can be used as directly detectable labels include fluorescein, tetramethylrhodamine, Texas Red, Oregon Green®, and a number of others (e.g., Haugland, Handbook of Fluorescent Probes-9th Ed., 2002, Molec. Probes, Inc., Eugene OR; Haugland, The Handbook: A Guide to Fluorescent Probes and Labeling Technologies-10th Ed., 2005, Invitrogen, Carlsbad, CA). Also included are light-emitting or otherwise detectable dyes. The light emitted by the dyes can be visible light or invisible light, such as ultraviolet or infrared light. In exemplary embodiments, the dye may be a fluorescence resonance energy transfer (FRET) dye; a xanthene dye, such as fluorescein and rhodamine; a dye that has an amino group in the alpha or beta position (such as a naphthylamine dye, 1-dimethylaminonaphthyl-5-sulfonate, 1-anilino-8-naphthalende sulfonate and 2-p-touidinyl-6-naphthalene sulfonate); a dye that has 3-phenyl-7-isocyanatocoumarin; an acridine, such as 9-isothiocyanatoacridine and acridine orange; a pyrene, a bensoxadiazole and a stilbene; a dye that has 3-(ε-carboxypentyl)-3′-ethyl-5,5′-dimethyloxacarbocyanine (CYA); 6-carboxy fluorescein (FAM); 5&6-carboxyrhodamine-110 (R110); 6-carboxyrhodamine-6G (R6G); N,N,N′,N′-tetramethyl-6-carboxyrhodamine (TAMRA); 6-carboxy-X-rhodamine (ROX); 6-carboxy-4′,5′-dichloro-2′,7′-dimethoxyfluorescein (JOE); ALEXA FLUOR™; Cy2; Texas Red and Rhodamine Red; 6-carboxy-2′,4,7,7′-tetrachlorofluorescein (TET); 6-carboxy-2′,4,4′,5′,7,7′-hexachlorofluorescein (HEX); 5-carboxy-2′,4′,5′,7′-tetrachlorofluorescein (ZOE); NAN; NED; Cy3; Cy3.5; Cy5; Cy5.5; Cy7; and Cy7.5; IR800CW, ICG, Alexa Fluor 350; Alexa Fluor 488; Alexa Fluor 532; Alexa Fluor 546; Alexa Fluor 568; Alexa Fluor 594; Alexa Fluor 647; Alexa Fluor 680, or Alexa Fluor 750.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), alpha-naphtol pyronin (.alpha.-NP), o-dianisidine (OD), 5-bromo-4-chloro-3-indolylphosp-hate (BCIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for alkaline phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Exemplary iodine-based labels include diatrizoic acid (Hypaque®, GE Healthcare) and its anionic form, diatrizoate. Diatrizoic acid is a radio-contrast agent used in advanced X-ray techniques such as CT scanning. Also included are iodine radioisotopes, described below.

Examples of luminescent labels include luminol, isoluminol, acridinium esters, 1,2-dioxetanes and pyridopyridazines. Examples of electrochemiluminescent labels include ruthenium derivatives

Examples of radioactive labels include radioactive isotopes of iodide, cobalt, selenium, tritium, carbon, sulfur and phosphorous. Exemplary radioisotopes that can be used as detectable labels include ³²p, ³³P, ³⁵S, ³H, ¹⁸F, ¹¹C, ¹³N, ¹⁵O, ¹¹¹In, ¹⁶⁹Yb, ^{99m}TC, ⁵⁵Fe, and isotopes of iodine such as ¹²³I, ¹²⁴I, ¹²⁵I, and ¹³¹I. These radioisotopes have different half-lives, types of decay, and levels of energy which can be tailored to match the needs of a particular protocol.

Nanoparticles usually range from about 1-1000 nm in size and include diverse chemical structures such as gold and silver particles and quantum dots. When irradiated with angled incident white light, silver or gold nanoparticles ranging from about 40-120 nm will scatter monochromatic light with high intensity. The wavelength of the scattered light is dependent on the size of the particle. Four to five different particles in close proximity will each scatter monochromatic light, which when superimposed will give a specific, unique color. Derivatized nanoparticles such as silver or gold particles can be attached to a broad array of molecules including, proteins, antibodies, small molecules, receptor ligands, and nucleic acids. Specific examples of nanoparticles include metallic nanoparticles and metallic nanoshells such as gold particles, silver particles, copper particles, platinum particles, cadmium particles, composite particles, gold hollow spheres, gold-coated silica nanoshells, and silica-coated gold shells. Also included are silica, latex, polystyrene, polycarbonate, polyacrylate, PVDF nanoparticles, and colored particles of any of these materials.

Quantum dots are fluorescing crystals about 1-5 nm in diameter that are excitable by light over a large range of wavelengths. Upon excitation by light having an appropriate wavelength, these crystals emit light, such as monochromatic light, with a wavelength dependent on their chemical composition and size. Quantum dots such as CdSe, ZnSe, InP, or InAs possess unique optical properties; these and similar quantum dots are available from a number of commercial sources (e.g., NN-Labs, Fayetteville, AR; Ocean Nanotech, Fayetteville, AR; Nanoco Technologies, Manchester, UK; Sigma-Aldrich, St. Louis, MO).

Detectable labels may be linked to the antibodies described herein or to any other molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents or antibodies, etc. Moreover, the skilled artisan would appreciate that each additional binding agent or antibody used to characterize a biological marker of interest may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

The antibodies, or antigen-binding fragments thereof, can be used in any of the compositions, methods, and/or kits described herein.

### Methods of Use

Embodiments of the present invention include methods of using the antibodies, and antigen-binding fragments thereof, described herein to determine the presence, absence, amount, levels, and/or subcellular localization of an NRP2 polypeptide in a biological sample, such as a biological sample. Such methods include, for example, the use of the antibodies, or antigen-binding fragments thereof, as a companion diagnostic to identify a subject for NRP2-targeted therapy, including for the administration of at least one NRP2-targeted therapeutic agent. In some embodiments, the subject is considered suitable for NRP2-targeted therapy if the amount of NRP2 polypeptide in a biological sample is increased relative to a control. In some embodiments, the subject is considered suitable for NRP2-targeted therapy if the subcellular localization of the NRP2 polypeptide is increased relative to a control, for example, subcellular localization to the nucleus or nuclear envelope. In particular embodiments, the methods include using the antibodies, or antigen-binding fragment thereof, as a companion diagnostic to identify a subject having or suspected of having a cancer or tumor that expresses or over-expresses an NRP2 polypeptide, and optionally for treating or causing the subject to be treated with an NRP2-targeted therapy.

Certain embodiments relate generally to methods of determining an amount of a human NRP2 polypeptide in a biological sample, comprising (a) contacting the biological sample with an antibody, or antigen-binding fragment thereof, as described herein; and (b) determining the amount of the antibody, or antigen-binding fragment thereof, in the biological sample, which thereby determines the amount of the human NRP2 polypeptide in the biological sample. In certain embodiments, the sample is denatured prior to or during step (a) of the methods.

Also included are methods of identifying an NRP2-expressing cancer in a biological sample of cancer tissue from a subject, comprising (a) contacting the biological sample with an antibody, or antigen-binding fragment thereof, as described herein; and (b) determining the amount or subcellular localization of the antibody, or antigen-binding fragment thereof, in the biological sample, which thereby determines the amount or subcellular localization of NRP2 in the biological sample; and (c) identifying the NRP2-expressing cancer if (i) the amount of NRP2 in the biological sample of cancer tissue from the subject is increased relative to a control or reference or (ii) the subcellular localization of the NRP2 is increased relative to a control or reference. In certain embodiments, the increase in subcellular localization (e.g., relative to cell surface localization) is to the nucleus or nuclear envelope. That is, in specific embodiments, the subcellular localization of the NRP2 is increased in the nucleus or nuclear envelope relative to the control or reference. In some embodiments, (b) comprises determining the ratio of NRP2 localized in the nucleus or nuclear envelope (nuclear NRP2) relative to NRP2 localized on the cell surface (cell surface NRP2), and (c) comprises identifying the NRP2-expressing cancer if the ratio of nuclear NRP2/cell surface NRP2 is increased relative to a control or reference. Some embodiments include administering or causing to be administered to the subject having the NRP2-expressing cancer of (c) at least one NRP2-targeted therapeutic agent, for example, a therapeutic antibody, or antigen-binding fragment thereof, which binds to human NRP2, either as a standalone agent or in combination with at least one additional anti-cancer therapy or agent such as one or more chemotherapeutic agents.

Also included are methods of identifying a subject for NRP2-targeted therapy, comprising (a) contacting a biological sample from the subject with an antibody, or antigen-binding fragment thereof, as described herein; (b) determining the amount or subcellular localization of the antibody, or antigen-binding fragment thereof, in the biological sample, which determines the amount or subcellular localization of NRP2 in the sample; and (c) identifying the subject as suitable for NRP2-targeted therapy if (i) the amount of the NRP2 in the biological sample is increased relative to a control or reference, or (ii) the subcellular localization of the NRP2 is increased relative to a control or reference. In some embodiments, the subcellular localization of the NRP2 is increased in the nucleus or nuclear envelope relative to the control or reference. In specific embodiments, step (b) comprises determining the ratio of NRP2 localized in the nucleus or nuclear envelope (nuclear NRP2) relative to NRP2 localized on the cell surface (cell surface NRP2), and (c) comprises identifying the NRP2-expressing cancer if the ratio of nuclear NRP2/cell surface NRP2 is increased relative to a control or reference. Some embodiments include administering or causing to be administered to the subject of (c) at least one NRP2-targeted therapeutic agent, either as a standalone therapeutic or in combination with one or more additional therapeutic agents, for example, anti-cancer agents.

Also included are methods for identifying a treatment regimen for a subject with castration-resistant prostate cancer (CRPC) that is relapsed/refractory to androgen receptor (AR)-targeted therapy. “Relapse” refers to the recurrence of a past condition or disease state, for example, following a period of treatment-related dormancy or disease inactivity. A patient that is “refractory” to AR-targeted therapy does not significantly respond to, has previously failed to respond to, or has become non-responsive (e.g., via selection) to the AR-targeted therapy Here, it has been shown that increased nuclear localization of (sumoylated) NRP2 promotes castration-resistant prostate cancer (CRPC)-specific gene expression by stabilizing a complex between the androgen receptor (AR) and nuclear pore proteins (see, for example, Dutta et al., Oncogene. 41:3747-3760, 2022). Such subjects are typically considered suitable for a more aggressive treatment regimen than AR-targeted therapy, for example, an aggressive treatment regimen that includes one or more chemotherapeutic agents. Certain of these and related embodiments thus comprise (a) contacting a biological sample from the subject with an antibody, or antigen-binding fragment thereof, as described herein; (b) determining the subcellular localization of the antibody, or antigen-binding fragment thereof, in the biological sample, which determines the subcellular localization of NRP2 in the sample; and (c) identifying the subject as being suitable for an aggressive treatment regimen if the subcellular localization of the NRP2 in the nucleus or nuclear envelope is increased relative to a control or reference. In some embodiments, (b) comprises determining the ratio of NRP2 localized in the nucleus or nuclear envelope (nuclear NRP2) relative to NRP2 localized on the cell surface (cell surface NRP2), and (c) comprises identifying the subject as being suitable for the aggressive treatment regimen if the ratio of nuclear NRP2/cell surface NRP2 is increased relative to a control or reference. In specific embodiments, the aggressive treatment regimen comprises at least one NRP2-targeted therapeutic agent, for example, a therapeutic antibody, or antigen-binding fragment thereof, which binds to human NRP2, in combination with at least one chemotherapeutic agent, including DNA damaging agents.

Also included are more general methods of identifying NRP2-mediated drug resistance in a subject, comprising (a) contacting a biological sample from the subject with an antibody, or antigen-binding fragment thereof, as described herein; (b) determining the subcellular localization of the antibody, or antigen-binding fragment thereof, in the biological sample, which determines the subcellular localization of NRP2 in the sample; and (c) identifying the subject as having NRP2-mediated drug resistance if the subcellular localization of NRP2 to the nucleus or nuclear envelope is increased relative to a control or reference. Some embodiments include administering or causing to be administered to the subject of (c) at least one NRP2-targeted therapeutic agent, optionally a therapeutic antibody, or antigen-binding fragment thereof, which binds to human NRP2, optionally in combination with at least one additional anti-cancer therapy or agent, optionally radiotherapies, cancer immunotherapies, chemotherapeutic agents (optionally DNA damaging agents, DNA repair inhibitors), hormonal therapeutic agents, kinase inhibitors, anti-growth factor therapies, and androgen receptor (AR)-targeted therapies. In some embodiments, (c) comprises correlating a higher increase in subcellular localization of NRP2 to the nucleus or nuclear envelope with a more advanced stage of NRP2-mediated drug resistance.

In some embodiments, the NRP2-targeted therapeutic agent is a therapeutic antibody, or antigen-binding fragment thereof, which binds to human NRP2. Examples of such agents are described in WO 2019/195770 and WO 2021/067761. In some embodiments, the NRP2-targeted therapeutic agent is a small molecule inhibitor of NRP2. Examples of such agents include benzamidine-based NRP2 inhibitors (see, for example, Said et al., Bioorg Chem. 100:103856, 2020), and others. Examples of additional anti-cancer therapies and agents include radiotherapies, cancer immunotherapies or immunotherapy agents (e.g., immune checkpoint modulatory agents, cancer vaccines, oncolytic viruses, cytokines, cell-based immunotherapies), chemotherapeutic agents (for example, DNA damaging agents, DNA repair inhibitors), hormonal therapeutic agents (e.g., hormonal agonists, hormonal antagonists), kinase inhibitors, anti-growth factor therapies, and androgen receptor (AR)-targeted therapies.

In some embodiments, the at least one chemotherapeutic agent is selected from one or more of an alkylating agent, an anti-metabolite, a cytotoxic antibiotic, a topoisomerase inhibitor (type 1 or type II), and an anti-microtubule agent. In some embodiments:
the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);
the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);
the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;
the topoisomerase inhibitor is selected from one or more of camptothecin, irinotecan, topotecan, etoposide, doxorubicin, mitoxantrone, teniposide, novobiocin, merbarone, and aclarubicin; and/or
the anti-microtubule agent is selected from one or more of taxanes (optionally paclitaxel and docetaxel) and vinca alkaloids (optionally vinblastine, vincristine, vindesine, vinorelbine).

In some embodiments, the at least one hormonal therapeutic agent is a hormonal agonist or a hormonal antagonist. In some embodiments, the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog. In some embodiments, the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

In some embodiments, the kinase inhibitor is selected from one or more of adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

Particular examples of anti-cancer agents include PD-L1 inhibitors, PD-1 inhibitors, EGFR inhibitors, VEGF/VEGFR inhibitors, and androgen receptor (AR)-targeted therapies, including androgen deprivation therapies. Specific examples of PD-L1 inhibitors include atezolizumab, avelumab, and durvalumab; examples of PD-1 inhibitors include nivolumab, pembrolizumab, cemiplimab, dostarlimab, and retifanlimab, examples of EGFR inhibitors include cetuximab, dacomitinib, erlotinib, gefitinib, lapatinib, mobocertinib, necitumumab, neratinib, osimertinib, panitumumab, and vandetanib; examples of VEGF/VEGFR inhibitors include aflibercept, axitinib, bevacizumab, cabozantinib, lenvatinib, pazopanib, ramucirumab, regorafenib, sorafenib, sunitinib, tivozanib, and vandetanib. Specific examples of AR-targeted therapies include steroidal antiandrogens (SAAs) such as abiraterone acetate, allylestrenol, chlormadinone acetate, cyproterone acetate, delmadinone acetate, gestonorone caproate, hydroxyprogesterone caproate, medroxyprogesterone acetate, megestrol acetate, osaterone acetate, oxendolone, and spironolactone; and nonsteroidal antiandrogens (NSAAs) such as aminoglutethimide, apalutamide, bicalutamide, enzalutamide, flutamide, ketoconazole, nilutamide, and topilutamide.

Certain embodiments comprise first obtaining the biological sample from a subject, for example, by receiving the biological sample from a healthcare provider, including wherein the subject has or is suspected of having an NRP2-associated disease, or a cancer. In some embodiments, step (a) comprises receiving the biological sample from a healthcare provider, such as a physician, clinic, or hospital. The entity receiving the biological sample and/or performing the testing can be associated with or separate from the healthcare provider. In particular embodiments, the entity receiving the sample and/or performing the testing is a third party diagnostic company. In some embodiments, the entity receiving the sample and/or performing the testing is a clinical or hospital-associated diagnostic laboratory. In some embodiments, the method comprises providing information to the same or different healthcare provider (e.g., physician) on the presence, absence, amount, levels, or subcellular localization of the NRP2 polypeptide in the biological sample. In some embodiments, the information on the subcellular localization of NRP2 comprises a ratio of nuclear NRP2/cell surface NRP2 in the biological sample.

Certain embodiments, for example, wherein the amount or subcellular localization of the NRP2 in the biological sample is increased relative to a control or reference, comprise identifying the subject has having an “NRP2-associated disease or condition”, which refer to diseases and conditions in which NRP2 activity, expression, and/or spatial distribution plays a role in the pathophysiology of that disease or condition. Exemplary NRP2-associated diseases and conditions include cancer and diseases or pathologies associated with cancer including cancer cell growth, cancer initiation, cancer migration, cancer cell adhesion, invasion, chemoresistance, and metastasis. Further examples of NRP2-associated diseases and conditions include inflammatory diseases or conditions such as rheumatoid arthritis (RA), osteoarthritis (OA), and inflammatory lung diseases (ILDs) such as sarcoidosis, scleroderma, chronic obstructive pulmonary disorder (COPD), and pulmonary fibrosis. Additional examples include diseases associated with lymphatic development, lymphangiogenesis, and lymphatic damage, including edema, lymphedema, secondary lymphedema, inappropriate fat absorption and deposition, excess fat deposition, and vascular permeability. Also included are diseases associated with infections including latent infections, and diseases associated with allergic disorders/diseases and allergic responses, including neutrophilic asthma, antineutrophil cytoplasmic antibody (ANCA)-associated systemic vasculitis, systemic lupus erythematosus, inflammasome-related disease(s), and skin-related neutrophil-mediated disease(s) such as pyoderma gangrenosum. Additional examples include diseases associated with granulomatous inflammatory diseases including sarcoidosis and granulomas, and fibrotic diseases including endometriosis, fibrosis, endothelial to mesenchymal transition (EMT), and wound healing, among others. Also included are diseases associated with inappropriate smooth muscle contractility, smooth muscle compensation and decompensation, vascular smooth muscle cell migration and/or adhesion, and diseases associated with inappropriate autophagy, phagocytosis, and efferocytosis. Also included are diseases associated with inappropriate migratory cell movement, as described herein. Additional examples include neuronal diseases, including diseases associated with peripheral nervous system remodeling and pain perception. Also included are diseases associated with bone development and/or bone remodeling. Typically, the term “inappropriate” refers to an activity or characteristic that associates with or causes a pathology or disease state.

The biological sample can include any variety of samples, fluids, or tissues. Non-limiting examples of biological samples include blood, plasma, skin, hair, hair follicles, saliva, oral mucous, vaginal mucous, sweat, tears, epithelial tissues, urine, semen, seminal fluid, seminal plasma, prostatic fluid, excreta, biopsy, ascites, cerebrospinal fluid, synovial fluid, bronchoalveolar lavage (BALF), lymph, tissue extracts sample, and biopsy samples, such as cancer or tumor biopsy sample, or a suspected cancer or tumor biopsy sample.

In some embodiments, the biological sample is a biopsy sample, including wherein the biopsy sample is a cancer or suspected cancer biopsy sample. In some embodiments, the biopsy sample is selected from skin tissue, liver tissue, pancreatic tissue, prostate tissue, mesothelial tissue, epithelial tissue, ovarian tissue, colorectal tissue, gastric tissue, brain tissue, lung tissue, kidney tissue, bladder tissue, uterine tissue, esophageal tissue, cervical tissue, testicular tissue, breast tissue, and mesenchymal tissue such as bone tissue, cartilage tissue, fat tissue, muscle tissue, vascular tissue, blood, or hematopoietic cells/tissue, for example, a liquid biopsy.

In some embodiments, the control or reference is a reference standard, a biological sample from a healthy subject, or a healthy biological sample from the same subject. In specific embodiments, the control is a non-cancerous biological sample from the same subject, for example, of the same tissue type.

The presence, absence, amount, levels, or cellular localization (e.g., surface, subcellular) of an antibody, or an antigen-binding fragment thereof, and thus an NRP2 polypeptide in a biological sample can be measured according to any variety of techniques in the art. For instance, certain embodiments may employ standard methodologies and detectors. Examples include immunohistochemistry (IHC), immunofluorescence (IF), Western blotting, immunoprecipitation, enzyme-linked immunosorbent assays (ELISA), slot blotting, and peptide mass fingerprinting. Certain embodiments may employ cell-sorting or cell visualization or imaging devices/techniques to visualize, detect, and/or quantitate the amount or levels of an antibody, or antigen-binding fragment thereof. Examples include flow cytometry (or FACS), immunofluorescence analysis (IFA), and in situ hybridization techniques, such as fluorescent in situ hybridization (FISH).

Specific embodiments include the use of IHC or IF analysis. Examples include chromogenic immunohistochemistry (CIH), for example, wherein the anti-NRP2 antibody is conjugated to an enzyme, such as peroxidase (immunoperoxidase), which catalyzes a color-producing reaction (see, for example, Ramos-Vara, “Technical Aspects of Immunohistochemistry”. Veterinary Pathol. 42:405-426, 2005). In IF analysis, the antibody is labeled directly or indirectly with a fluorophore, such as fluorescein or rhodamine, which allows visualization by light microscopy with a fluorescent microscope. Certain embodiments employ primary (or direct) IF analysis, wherein the primary anti-NRP2 antibody is chemically linked to a fluorophore. Certain embodiments employ secondary (or indirect) IF analysis, which requires at least two antibodies; the unlabeled primary anti-NRP2 antibody specifically binds the target molecule, and one or more secondary antibodies, which carries the fluorophore(s), bind to the primary antibody.

Thus, in certain embodiments, steps (a) and/or (b) of the methods provided herein comprise performing an IHC or IF assay on the biological sample. Some embodiments include the step(s) of preparing the biological sample, for example, by any combination of tissue collection, fixation (e.g., formalin), embedding in a medium such as paraffin wax or cryomedia, and sectioning with an instrument such as a microtome, cryostat, or vibratome (for example, to a size range of about 3-5 μm). Certain embodiments include the step(s) of mounting the sample (e.g., slices) on slides, dehydrating the sample using alcohol washes of increasing concentrations (e.g., 50%, 75%, 90%, 95%, 100%), and clearing the sample using a detergent (e.g., xylene), and imaging under a microscope. Certain embodiments include the additional pre-antibody treatment steps of deparaffinization and antigen retrieval. For instance, for certain formalin-fixed paraffin-embedded tissues, antigen-retrieval includes pre-treating the sections with heat or protease. Also included are any appropriate wash steps. Particular embodiments include the step of imaging or visualizing the treated biological sample for the presence, absence, levels, amount, or localization (e.g., surface, subcellular) of the labeled/stained anti-NRP2 antibody, as described herein.

In specific embodiments, the IHC or IF assay comprises a multiplex IHC or IF assay (see, for example, Tan, Wei Chang Colin et al. “Overview of multiplex immunohistochemistry/immunofluorescence techniques in the era of cancer immunotherapy.” Cancer communications (London, England) vol. 40, 4 (2020): 135-153). Multiplex IHC or IF includes contacting the biological sample with at least one additional antibody, or antigen-binding fragment thereof, which specifically binds to an additional marker of interest, which has or utilizes a different (direct or indirect) detectable label. In particular embodiments, the additional marker of interest is selected from one or more of signal transduction pathway molecules (for example, VEGF-C, VEGF-A, EGF, IGF, FGF, TGF-beta, VEGFR1, VEGFR2, VEGFR3, CCR7, EGFR1, EGFR2, PDGFR, TGFR1, TGFR2, TGFR3, c-MET); EMT markers such as mesenchymal markers (for example, N-cadherin, E-cadherin, OB-cadherin, ZO-1, a5B1 integrin 1, αVβ6 integrin, Syndecan-1, FSP1, Cytokeratin, α-SMA, Vimentin 1, β-Catenin), epithelial markers (for example, CDH1, EPCAM, claudins, and cytokeratins), and related transcription factors (for example, Snail, Slug, ZEB1, ZEB2, and Twist); lymphangiogenesis markers (for example, lymphatic vessel endothelial hyaluronan receptor-1, or LYVE-1); fibrosis markers (for example, collagen fibers, matricellular proteins such as tenascin-C, α-SMA); and immune activation/exhaustion markers and immune modulators (for example, CD45RA, CD45RO, CD27, CD62L, CD95, PD-1, PD-L1, CD80, CD86, CXCR4, BLC, sCD30, MCP-2, IP-10, APRIL, SIL-2R, IL7, MIF, MIP-1b, SCF, SDF-1a, sTNF-RI).

Certain embodiments employ conventional biology methods, software, and systems for diagnostic purposes. Computer software products or method typically include or use computer readable medium having computer-executable instructions for performing the logic steps of the method. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Quelette and Bzevanis Bioinformatics: A Practical Guide for Analysis of Gene and Proteins (Wiley & Sons, Inc., 2nd ed., 2001). See also U.S. Pat. No. 6,420,108.

### Expression and Purification Systems

Certain embodiments include methods and related compositions for expressing and purifying an anti-NRP2 antibody or other polypeptide-based agent described herein. Such recombinant anti-NRP2 antibodies can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, anti-NRP2 antibodies may be prepared by a procedure including one or more of the steps of: (a) preparing a construct comprising a polynucleotide sequences that encode an anti-NRP2 antibody heavy and light chain and that are operably linked to a regulatory element; (b) introducing the constructs into a host cell; (c) culturing the host cell to express an anti-NRP2 antibody; and (d) isolating an anti-NRP2 antibody from the host cell.

Anti-NRP2 antibody polynucleotides are described elsewhere herein. In order to express a desired polypeptide, a nucleotide sequence encoding an anti-NRP2 antibody, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORTI plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments may employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21 (DE3), a ADE3 lysogen of BL21 that supports T7-mediated expression and is deficient in lon and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS.TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15 L, 50 L, 100 L, and 200 L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., PNAS. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5:Unit5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216(1980)); and myeloma cell lines such as NSO and Sp2/0. For a review of certain mammalian host cell lines suitable for antibody production, see, e.g., Yazaki and Wu, Methods in Molecular Biology, Vol. 248 (B. K. C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., PNAS USA. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009). These and related embodiments can be used, for example, to generate microarrays of anti-NRP2 antibodies which can then be used for screening libraries to identify antibodies and antigen-binding domains that interact with the NRP2 polypeptide(s) of interest.

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. Certain specific embodiments utilize serum free cell expression systems. Examples include HEK293 cells and CHO cells that can be grown in serum-free medium (see, e.g., Rosser et al., Protein Expr. Purif. 40:237-43, 2005; and U.S. Pat. No. 6,210,922).

An antibody, or antigen-binding fragment thereof, produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6×His), cMyc tags, V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating anti-NRP2 antibodies and antigen-binding fragments thereof, and composition comprising concentrated soluble proteins. In different aspects such concentrated solutions of anti-NRP2 antibodies may comprise proteins at a concentration of about 5 mg/mL; or about 8 mg/mL; or about 10 mg/mL; about 15 mg/mL; or about 20 mg/mL.

In some aspects, such compositions may be substantially monodisperse, meaning that an at least one anti-NRP2 antibody exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, the reagents, anti-NRP2 antibodies, or related agents have a purity of at least about 90%, as measured according to routine techniques in the art. In certain embodiments, such as diagnostic compositions, an anti-NRP2 antibody composition has a purity of at least about 95%. In specific embodiments, an anti-NRP2 antibody composition has a purity of at least about 97% or 98% or 99%. In other embodiments, such as when being used as reference or research reagents, anti-NRP2 antibodies can be of lesser purity, and may have a purity of at least about 50%, 60%, 70%, or 80%. Purity can be measured overall or in relation to selected components, such as other proteins, for example, purity on a protein basis.

Purified anti-NRP2 antibodies can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more canonical or non-canonical biological activities can be measured according to cell-based assays, including those that utilize a cellular binding partner of a selected anti-NRP2 antibody, which is functionally coupled to a readout or indicator, such as a fluorescent or luminescent indicator of biological activity, as described herein.

In certain embodiments, an anti-NRP2 antibody composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15:1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade proteins such as protein fragments and antibodies can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents (e.g., anti-NRP2 antibodies, and antigen-binding fragments) are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of anti-NRP2 antibodies or variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Anti-NRP2 antibodies with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains an anti-NRP2 antibody of the present disclosure.

### Compositions and Kits

Also included are compositions that comprise the antibodies, or antigen-binding fragments thereof, described herein, optionally in combination with a suitable carrier. A composition or carrier may be liquid, semi-liquid, semi-solid, or solid.

Solutions or suspensions may include, for example, a sterile diluent (such as water), saline solution (e.g., phosphate buffered saline (PBS), physiological saline, Ringer's solution, isotonic sodium chloride), fixed oil, polyethylene glycol, glycerin, propylene glycol or other synthetic solvent; antimicrobial agents (such as benzyl alcohol and methyl parabens); antioxidants (such as ascorbic acid and sodium bisulfite), chelating agents (such as ethylenediaminetetraacetic acid (EDTA)); and/or buffers (such as acetates, citrates, phosphates, and other organic acids), including combinations of the foregoing. Also included as suitable carriers are solutions containing thickening and solubilizing agents, such as glucose, polyethylene glycol, polypropylene glycol and mixtures thereof. A surfactant may be added to facilitate the formation of a homogeneous solution or suspension. Surfactants are compounds that non-covalently interact with the antibody, or antigen-binding fragment thereof, so as to facilitate dissolution or homogeneous suspension of the conjugate in the aqueous system.

Additional examples of carriers include low molecular weight (e.g., less than about 10 residues) polypeptides or peptides; proteins such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, the antibody, or antigen-binding fragment thereof, is entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other diagnostic agents, such as detectable entities.

In particular embodiments, the antibody, or antigen-binding fragment thereof, is a freeze-dried or lyophilized, cryodesiccated. These terms refer to a dehydration process of freezing the antibody composition and then reducing the surrounding pressure to allow the frozen water in the composition to sublimate directly from the solid phase to the gas phase. Also included are solid compositions such as powders, granules, compressed tablets, pills, capsules, and the like. In some embodiments, solid composition contain one or more inert diluents or edible carriers. In certain embodiments, one or more of the following may be present: binders such as carboxymethylcellulose, ethyl cellulose, microcrystalline cellulose, gum tragacanth or gelatin; and excipients such as starch, lactose or dextrins, disintegrating agents such as alginic acid, sodium alginate, Primogel, corn starch and the like.

Certain embodiments include kits, comprising one or more of the anti-NRP2 antibodies, or antigen-binding fragments thereof, as described herein, optionally in one or more containers. The kits can include written instructions on how to use and/or prepare the antibodies for use, for example, as detection and/or diagnostic agents. In some embodiments, the written instructions describe how to use the antibodies, or antigen-binding fragments thereof, to identify a subject for arginine deprivation therapy, e.g., with arginine depletion agent(s). In some embodiments, the kit comprises material(s) for an IHC (e.g., chromogenic IHC) assay or an IF assay. In some embodiments, the kit comprises material(s) for an enzyme-linked immunosorbent assay (ELISA) or similar assay, for example, where the antibody, or antigen-binding fragment thereof, is attached or attachable to a solid substrate for performing an ELISA or similar assay.

The kits herein may also include a one or more additional therapeutic agents or other components suitable or desired for the indication being treated, or for the desired diagnostic application. An additional therapeutic agent may be contained in a second container, if desired. Examples of additional therapeutic agents include NRP2-targeted therapeutic agents, for example, a therapeutic antibody, or antigen-binding fragment thereof, which binds to human NRP2 (see, for example, WO 2019/195770; and WO 2021/067761).

Although the foregoing embodiments have been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Generation of Anti-Human NRP2 Antibodies

Antibody generation. Anti-NRP2 antibodies suitable for immunohistochemistry and related uses were generated by immunizing mice via an IP administration with 1×10⁶ Expi293 cells stably over expressing human NRP2A variant 2 (Origene Technologies Cat #RC220706), (prepared as more fully described below) using standard methodologies. Titers were boosted via S.C. administration of 10 ug/mouse of the corresponding recombinant NRP2 polypeptides listed in Table N1, using either IFA or Magic Mouse as the adjuvant. Mice were boosted every 2-3 weeks and then screened for initial titer and specificity using the NRP2 polypeptides listed in Table N1.

For all antibodies, spleens were isolated from immunized animals and fusion with mouse myeloma cells was performed to generate hybridomas using standard techniques. Fusion, plating into 96-well plates, ELISA screening of hybridomas, expansion and characterization of positive hybridomas (titer and isotype) and freezing of up to 15 hybridomas per antigen, was performed at The Scripps Research Institute (TSRI) Center for Antibody Development and Production. Antibody variable domain sequences were obtained using standard sequencing approaches performed at Lake Pharma, and are provided in Table S1 and Table S2. Recombinant antibodies were produced from hybridoma cells after expansion and purified from conditioned medium starting at 2 weeks of culture by flowing over a Protein A affinity column, eluting and storing in Phosphate Buffered Saline (1×PBS), pH 7.4. Each lot was tested for protein concentration, purity and endotoxin level. Purity by SDS-PAGE was routinely >90%.

### Example 2

### Characterization of Anti-Human NRP2 Antibodies

Initial assessments of binding affinity were completed using an enzyme-linked immunosorbent assay (ELISA), and then via western blotting to identify antibodies showing good binding to denatured epitopes. After initial identification as a strong binder, additional batches of antibody aNRP2-36v2 were prepared for additional characterization, as described below.

Antibody aNRP2-36v2 was tested for its specificity for binding to NRP2a via ELISA using three different purification lots. Amino acids 23-855 of NRP2v2 (SEQ ID NO: 14) were cloned with the native signal peptide and a C-terminal Myc & 6×His tag, produced in Expi293 cells, and purified by nickel affinity. A clear flat-bottom Immuno 96W plate (ThermoFisher, #14-245-153) was coated with the NRP2v2 protein at 2 ug/mL diluted in 1×PBS pH 7.4 (ThermoFisher, #10010031). Plate was coated overnight at 4° C. without shaking.

The next day, plate was washed 3 times with 300 uL/well of PBST and the plate was blocked with 100 μL of Casein (ThermoFisher, #37528) for 1 hour at room temperature with shaking (400 rpm). While the plate was incubated, the three lots of aNRP2-36v2 were diluted to 1 ug/mL in 1% BSA/PBS (Sigma, #A6003-25G) and titrated the proteins 4-fold in a 7-point titration with a 1% BSA/PBS blank well. The plate was washed 3 times with 300 uL/well of PBST and the titration of aNRP2-36v2 protein lots were added to the plate at 50 uL/well. The plate was incubated for 1 hour at room temperature with shaking (400 rpm). The plate was washed 3 times with 300 uL/well of PBST and the HRP conjugated goat anti-mouse IgG secondary antibody (Jackson Immuno, #115-035-071) was applied at a 1:5000 dilution in Casein at 50 uL/well. Plate was incubated for 1 hour at room temperature with shaking (400 rpm). The plate was washed 3 times with 300 uL/well of PBST and the plate was developed with Ultra TMB ELISA substrate (ThermoFisher, #PI34029) at 50 uL/well for 7 min. The colorimetric reaction was stopped using 50 uL/well of Stop Solution (Biolegend, #423001). The signals were read on a BioTek plate reader at 450 nm (BioTek PowerWave HT).

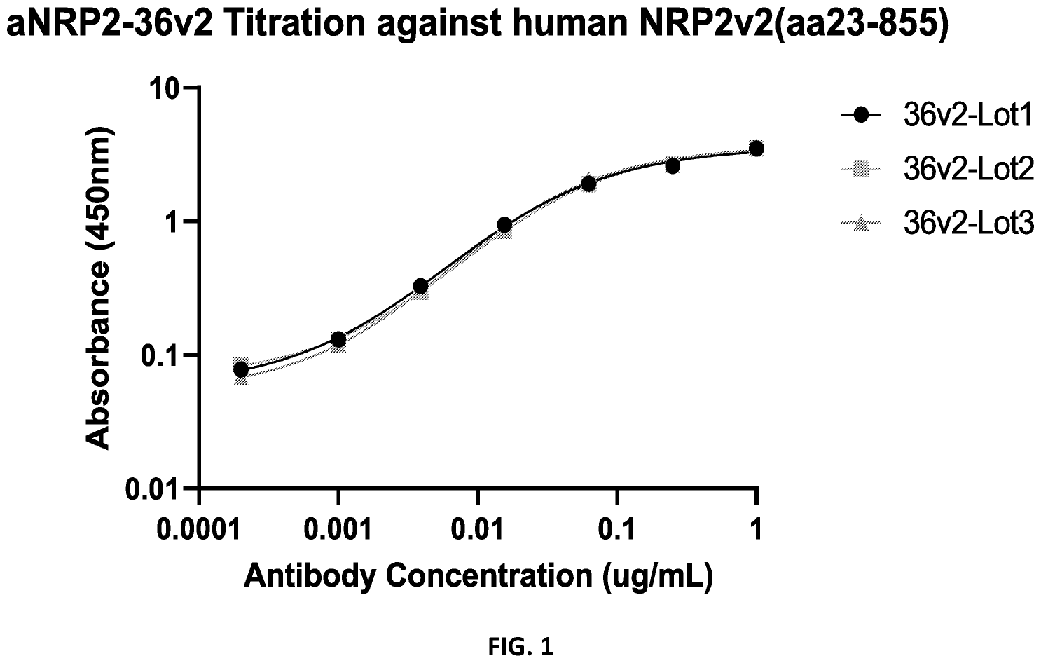

The results from this analysis (see FIG. 1) demonstrated that aNRP2-36v2 showed strong reproducible binding to the extracellular domains of NRP2.

### Example 3

### Epitope Mapping and Species Specificity Studies

Antibody aNRP2-36v2 was tested for its binding to different NRP2 domains and to evaluate its cross-reactivity to mouse, rat, and cyno NRP2, as well as testing for potential cross reactivity to human NRP1. Clear flat-bottom Immuno 96W plates (ThermoFisher, #14-245-153) were coated with various NRP2 proteins at 2 ug/mL diluted in 1×PBS pH 7.4 (ThermoFisher, #10010031). Three different batches of antibody aNRP2-36v2 were tested with proteins (SEQ ID NOs: 14, 15, 16, 17, 18, 19, 20, 21, 22, 23, 24, and 27; see Table N1). Plates were coated overnight at 4° C. without shaking. The next day, plates were washed 3 times with 300 uL/well of PBST and were blocked with 100 μL of Casein (ThermoFisher, #37528) for 1 hour at room temperature with shaking (400 rpm). While the plates were incubated, the three lots of aNRP2-36v2 to 1 ug/mL were diluted in 1% BSA/PBS (Sigma, #A6003-25G). The plate was washed 3 times with 300 uL/well of PBST and the 1 ug/mL solution of each aNRP2-36v2 antibody lot was added to the plate at 50 uL/well. The plate was incubated for 1 hour at room temperature with shaking (400 rpm). The plate was washed 3 times with 300 uL/well of PBST and the HRP conjugated goat anti-mouse IgG secondary antibody (Jackson Immuno, #115-035-071) was applied at a 1:5000 dilution in Casein at 50 uL/well. Plate was incubated for 1 hour at room temperature with shaking (400 rpm). The plate was washed 3 times with 300 uL/well of PBST and was developed with Ultra TMB ELISA substrate (ThermoFisher, #PI34029) at 50 uL/well for 7 min. The colorimetric reaction was stopped using 50 uL/well of Stop Solution (Biolegend, #423001). The signals were read on a BioTek plate reader at 450 nm (BioTek PowerWave HT).

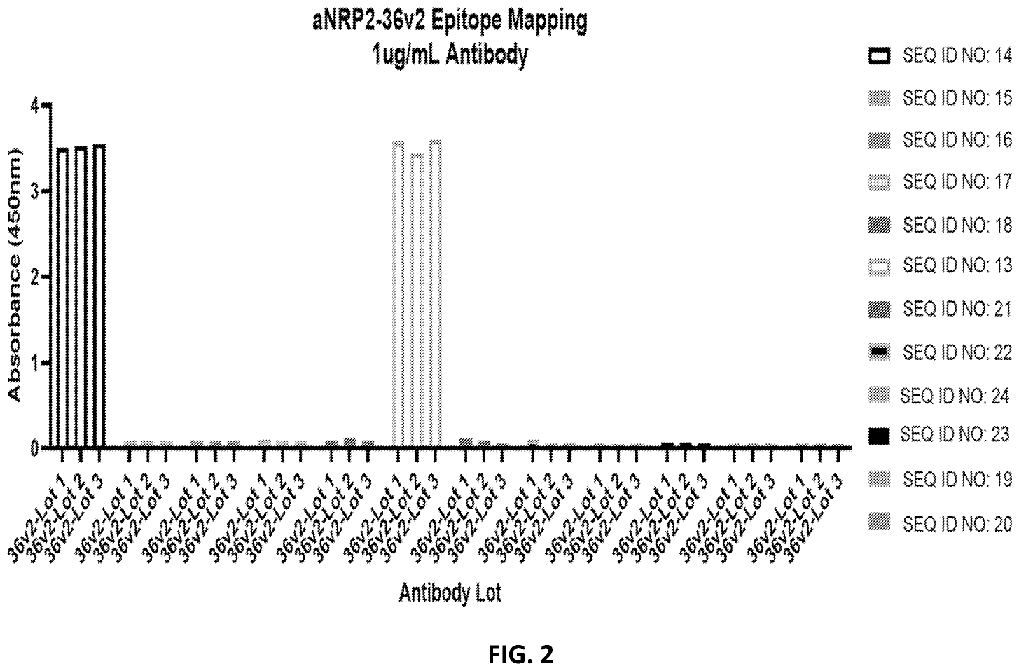

The results demonstrated (see FIG. 2) that antibody aNRP2-36v2 only binds to the NRP2 polypeptides NRP2v2 (23-855) and NRP2v5 (23-832), which indicates the epitope is located in NRP2 at residues 642-808, and recognizes both NRP2a and NRP2b. Antibody aNRP2-36v2 did not show any cross-reactivity with mouse, rat, or cyno NRP2, or with human NRP1. The three different lots also show similar magnitude of signals to each other which also indicates the consistency between the different purification lots.

### Example 4

### Characterization of Antibody Affinity and Binding Specificity

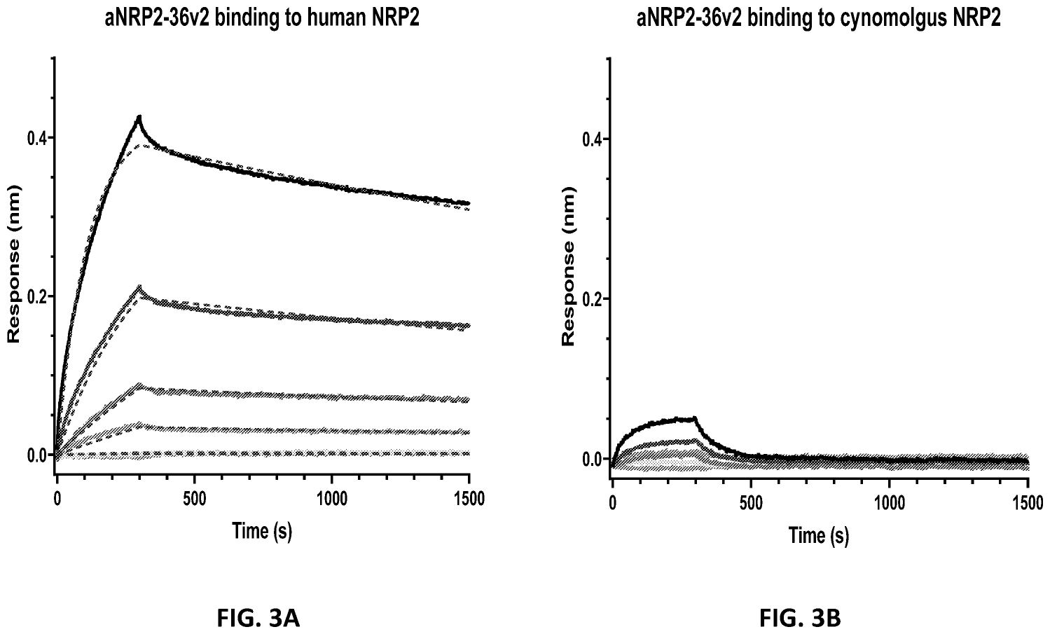

Biolayer interferometry (BLI) experiments were carried out on an Octet RED96e instrument (Sartorius). Antibody aNRP2-36v2 was tested for affinity towards human and cynomolgus NRP2. Antibodies and proteins were diluted in 1×PBS, 0.1% BSA, 0.02% Tween 20, pH 7.4. Octet Streptavidin biosensors (Sartorius, 18-5020) were immobilized with biotin-conjugated CaptureSelect anti-LC Kappa (murine) antibodies (Thermo Scientific, 7103152500). The biosensors were dipped into 1 ug/mL aNRP2-36v2 to capture the antibody on the biosensors. They were then dipped into human NRP2v2 (23-855) (SEQ ID NO: 14) or cyno NRP2×2 (23-854) (SEQ ID NO: 23) recombinant proteins varying in concentration from 1350 nM to 1.85 nM in a 3-fold dilution series. The resulting data was globally fit to a 1:1 interaction model using the Octet Data Analysis HT 11.1 software to obtain association (kₐ) and dissociation (k_{d}) rate constants and the resulting equilibrium binding constant (K_{D}). The results (see FIGS. 3A-3B) demonstrate that antibody aNRP2-36v2 shows strong binding to human NRP2 with a binding constant of about 9.7 nM, whereas it showed very weak binding to cynomolgus NRP2.

The epitope specificity of antibody aNRP2-36v2 binding to NRP2 was tested by incubation with a blocking peptide corresponding to residues 642-659 of NRP2 (SEQ ID NO: 25). This region was determined to be the likely epitope of aNRP2-36v2 based on its lack of binding to cynomolgus monkey NRP2 and an analysis of the sequence differences between human and cynomolgus NRP2 within region of NRP2 identified above. The region surrounding residue 651 of NRP2 was considered to be the most likely epitope location because of both the existence of amino acid differences in this region, and the existence of nearby flanking Cys residues which could account for the observed sensitivity of antibody binding to the redox state of NRP2 measured under either reducing or oxidized conditions as shown below.

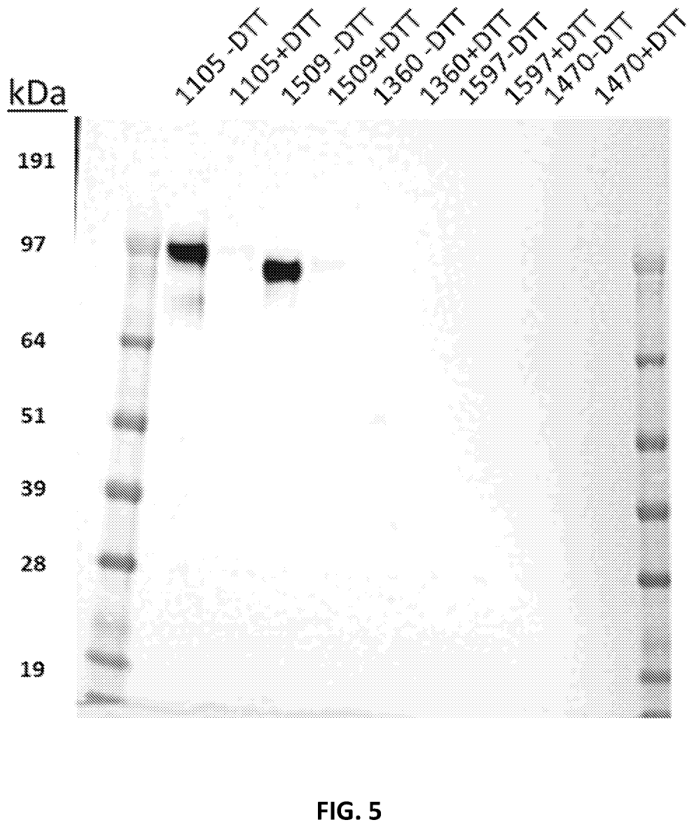

To determine if the peptide would block the interaction between aNRP2-36v2 and NRP2, aNRP2-36v2 was captured on BLI biosensors and dipped into NRP2 incubated with increasing amounts of peptide. Antibodies and proteins were diluted in 1×PBS, 0.1% BSA, 0.02% Tween 20, pH 7.4. Octet Streptavidin biosensors (Sartorius, 18-5020) were immobilized with biotin-conjugated CaptureSelect anti-LC Kappa (murine) antibodies (Thermo Scientific, 7103152500). The biosensors were dipped into 1 ug/mL aNRP2-36v2 to capture the antibody on the biosensors. They were then dipped into 450 nM human NRP2v2 (23-855) mixed with peptide varying in concentration from 1500 nM to 6.173 nM in a 3-fold dilution series. The results (see FIG. 5) show that increasing the concentration of blocking peptide results in a dose dependent reduction of the binding of antibody to NRP2, indicating that the peptide is binding to the antibody and preventing its association with NRP2.

### Example 5

### Western Blotting Specificity

To characterize the binding specificity of antibody aNRP2-36v2 to different denatured isoforms of NRP2 its binding specificity was tested via western blot. In addition to assessing the relative binding to NRP2a and NRP2b, cross-reactivity to mouse and rat NRP2 were also evaluated, as well as non-specific binding to NRP1. The proteins tested via Western were NRP2v2 (23-855) (SEQ ID NO: 14), NRP2v5 (23-832) (SEQ ID NO: 27), mouse NRP2v2 (23-855) (SEQ ID NO: 21), rat NRP2×2 (23-854) (SEQ ID NO: 22), and NRP1v2 (22-602) (SEQ ID NO: 24).

25 ng of each recombinant protein was denatured for 10 minutes at 70° C. with 1×LDS (ThermoFisher, NP0007) with or without 50 mM DTT and separated on a 4-12% Bis-Tris Protein Gel (ThermoFisher, NP0335) in 1×MOPS buffer (ThermoFisher, NP000102). The gel was then transferred to a nitrocellulose membrane (Fisher, cat. #IB23001) using an iBlot2 (Fisher, cat. #IB21001) for 7 min at 20V. The membrane was blocked for 1 hour using a 5% milk 0.05% TBST solution, followed by 1.0 ug/mL of the three lots of aNRP2-36v2 antibody diluted in 5% milk 0.05% TBST. Antibody was incubated on the blots overnight at 4° C. with rocking. After overnight incubation 3 washes with 0.05% TBST for 5 minutes each were performed and goat-a-mouse HRP secondary (Jackson Immuno Research, 115-035-071) diluted at 1:5,000 in the 5% milk mixture was applied to the blots for 1 hour. This was followed by 3 more washes with 0.05% TBST for 5 minutes each. The membrane was incubated in SuperSignal West Femto Chemiluminescent (ThermoFisher, PI34096) for 30 seconds and imaged in a Syngene G: Box Chemi XX6.

The results (see FIG. 5) demonstrate that antibody aNRP2-36v2 only reacts to the non-reduced proteins NRP2v2 (23-855) (SEQ ID NO: 14) and NRP2v5 (23-832) (SEQ ID NO: 27), or the NRP2a and NRP2b isoform proteins. This data is consistent with the ELISA epitope mapping data. All antibody lots consistently bind the NRP2v2 (23-855) (SEQ ID NO: 14) and NRP2v5 (23-832) (SEQ ID NO: 27) proteins and do not show binding to mouse or rat NRP2, and also do not cross-react with NRP1.

The specificity of aNRP2-36v2 in Western blotting was also confirmed by the ability of the blocking peptide NRP2 (642-659) (SEQ ID NO: 25) to block binding. For these studies, 100 ng of NRP2v2 (23-855) (SEQ ID NO: 14) recombinant protein and 20 ug of total protein from cell lysates of Expi293 cells transiently expressing NRP2a isoform 2 (SEQ ID NO: 10) were denatured for 10 minutes at 70° C. with 1×LDS (ThermoFisher, NP0007) and separated on a 4-12% Bis-Tris Protein Gel (ThermoFisher, NP0335) in 1×MOPS buffer (ThermoFisher, NP000102). The gel was then transferred to a nitrocellulose membrane (Fisher, cat. #IB23001) using an iBlot2 (Fisher, cat. #IB21001) for 7 min at 20V. The membrane was blocked for 1 hour using a 5% milk 0.05% TBST solution, followed by 1 ug/mL of aNRP2-36v2 antibody with or without 5 ug/mL blocking peptide, diluted in 5% milk 0.05% TBST. A peptide corresponding to NRP2v2 (809-820) (SEQ ID NO: 26) was also used as a control. Antibody was incubated on the blots overnight at 4° C. with rocking. After overnight incubation 3 washes with 0.05% TBST for 5 minutes each were performed and goat-a-mouse HRP secondary (Jackson Immuno Research, 115-035-071) diluted at 1:5,000 in the 5% milk mixture was applied to the blots for 1 hour. This was followed by 3 more washes with 0.05% TBST for 5 minutes each. The membrane was incubated in SuperSignal West Femto Chemiluminescent (ThermoFisher, PI34096) for 30 seconds and imaged in a Syngene G: Box Chemi XX6.

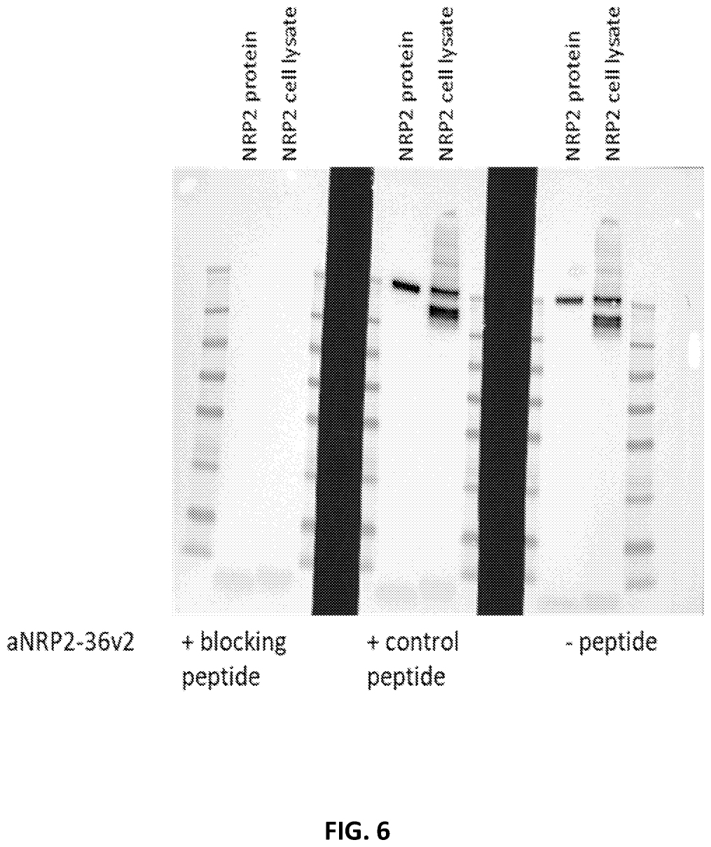

The results (see FIG. 6) demonstrate that the presence of the blocking peptide specifically prevents detection by aNRP2-36v2, indicating that the peptide is binding to the antibody and preventing its association with NRP2. These studies also demonstrates that the antibody is highly specific for NRP2 and does not recognize any other protein bands in cellular lysates.

### Example 6

### Tissue Staining

To evaluate the performance of aNRP2-36v2 in immunohistochemistry (IHC) analysis of tissues, sections were prepared from formalin fixed paraffin embedded (FFPE) tissue blocks containing skin biopsies from sarcoidosis patients. IHC tissue staining was developed in conjunction with the Muders laboratory at the University of Bonn, Germany.

Slides were de-paraffinized and rehydrated using xylol and sequential washing in decreasing alcohol series following standard procedures. Antigen retrieval was performed by heating slides in 1×TRS Citrate pH 6 (Dako, S236984-2) to 90° C. in a microwave and allowing to cool to room temperature. Slides were incubated with 0.3% hydrogen peroxide in PBS for 15 minutes at room temperature, then washed twice with PBS. A hydrophobic pen was used to encircle the tissues on the slides. Tissues were then blocked with 5% Bovine Serum Albumin (BSA) in PBS (Sigma Aldrich, A7906) for 25 minutes at room temperature. Primary antibodies (NBIC control antibody and aNRP2-36v2) were diluted in blocking buffer to 20 ug/ml and incubated on the tissue overnight at room temperature in a humidified chamber. For peptide blocking, aNRP2-36v2 was incubated for one hour with 100 ug/ml blocking peptide (SEQ ID NO: 25) prior to addition to the tissue.

The next day, slides were washed twice with PBS, and NRP2 was detected using the VECTASTAIN Elite ABC Peroxidase (HRP) kit (Vector Laboratories, PK-6102). Briefly, tissues were incubated with biotinylated goat anti-mouse IgG at a 1:200 dilution in PBS for 30 minutes at room temperature. Slides were washed twice with PBS and incubated with VECTASTAIN Elite ABC reagent (Avidin-HRP) for 30 minutes at room temperature. Slides were washed twice with PBS and NRP2 was visualized by incubating the tissues with ImmPACT NovaRED peroxidase substrate (Vector Laboratories, SK-4805) for 3 minutes. Reaction was quenched by rinsing in DI water, and counterstained with freshly filtered Harris Hematoxylin (Epredia Shandon, 6765001) for one minute. Slides were then rinsed under running DI water for 5 minutes, then incubated with Bluing Solution 1% lithium carbonate (Poly Scientific, S127) for one minute. Slides were rinsed again under running DI water for 5 minutes, then tissues were dehydrated by increasing alcohol series, followed by a xylene soak for 5 minutes. Finally, glass coverslips were mounted with Cytoseal Mountant XYL (Epredia, 8312-4) and tissues were imaged at 20× and 40×.

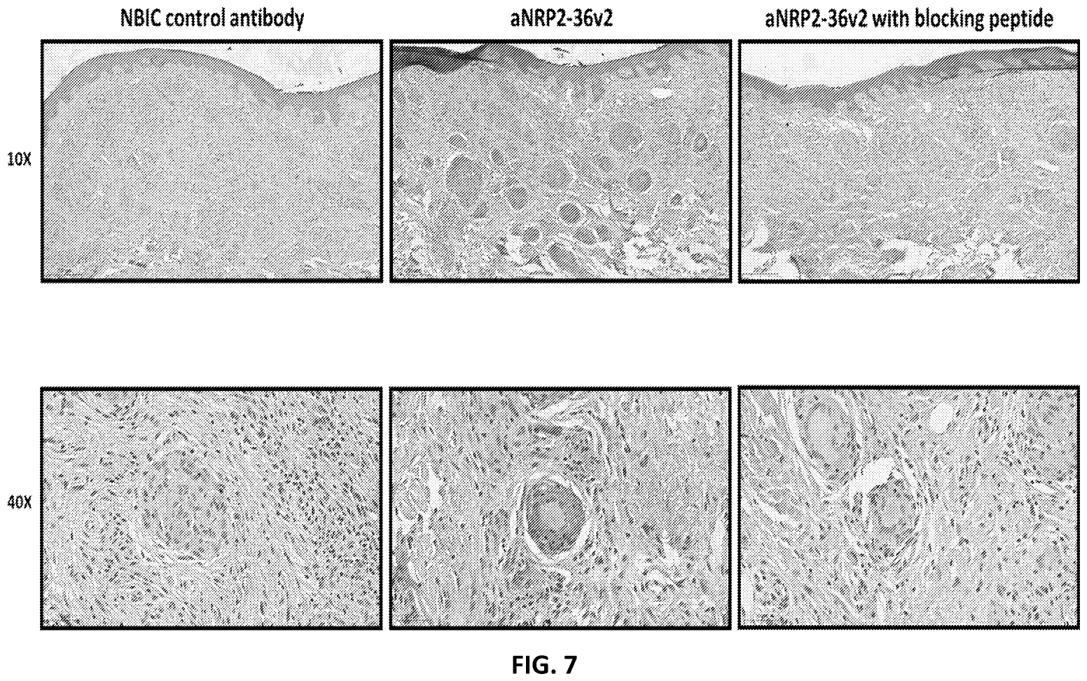

The images shown in FIG. 7 indicate that aNRP2-36v2 detects NRP2 protein expression in the skin of sarcoidosis patients and that NRP2 expression is concentrated within granulomas. Incubation of aNRP2-36v2 with blocking peptide prior to tissue staining prevented detection of NRP2 in sarcoidosis tissue, indicating that aNRP2-36v2 can specifically detect NRP2 in human FFPE tissue sections.

1. An isolated antibody, or an antigen-binding fragment thereof, which binds to a human neuropilin-2 (NRP2) polypeptide, and comprises
a heavy chain variable region (V_{H}) sequence that comprises complementary determining region V_{H}CDR1, V_{H}CDR2, and V_{H}CDR3 sequences set forth in SEQ ID NOs: 1-3, respectively;
and a light chain variable region (V_{L}) sequence that comprises complementary determining region V_{L}CDR1, V_{L}CDR2, and V_{L}CDR3 sequences, set forth in SEQ ID NOs: 4-6, respectively, including variants thereof having 1, 2, 3, 4, 5, or 6 total alterations across all of the CDR regions.

2. The isolated antibody, or antigen-binding fragment thereof, of claim 1, wherein the V_{H} sequence comprises an amino acid sequence that is at least 80, 85, 90, 95, 97, 98, 99, or 100% identical to SEQ ID NO: 7, and the V_{L} sequence comprises an amino acid sequence that is at least 80, 85, 90, 95, 97, 98, 99, or 100% identical to SEQ ID NO: 8.

3. The isolated antibody, or antigen-binding fragment thereof, of claim 1 or 2, which binds to a denatured form of the human NRP2 polypeptide.

4. The isolated antibody, or antigen-binding fragment thereof, of any one of claims 1-3, which binds to each of the human NRP2 polypeptides selected from human neuropilin-2a (NRP2a) variant 1 (v1), human NRP2a variant 2 (v2), human NRP2a variant 3 (v3), human neuropilin-2b (NRP2b) variant 4 (v4), and human NRP2b variant 5 (v5), optionally wherein the NRP2 polypeptides are selected from Table N1.

5. The isolated antibody, or antigen-binding fragment thereof, of any one of claims 1-4, which binds to the human NRP2 polypeptide at an epitope that comprises SEQ ID NO: 25.

6. The isolated antibody, or antigen-binding fragment thereof, of any one of claims 1-4, which binds to the human NRP2 polypeptide with an affinity of about or less than about 10 nM.

7. The isolated antibody, or antigen-binding fragment thereof, of any one of claims 1-6, which does not substantially bind to cynomolgus NRP2, mouse NRP2, rat NRP2, or human neuropilin-1 (NRP1).

8. The isolated antibody, or antigen-binding fragment thereof, of any one of claims 1-7, which comprises an IgA (including subclasses IgA1 and IgA2), IgD, IgE, IgG (including subclasses IgG1, IgG2, IgG3, and IgG4), or IgM Fc domain.

9. The antibody, or antigen-binding fragment thereof, of any one of claims 1-8, which is a monoclonal antibody.

10. The isolated antibody, or antigen-binding fragment thereof, of any one of claims 1-9, which comprises a mouse, rabbit, or goat IgG Fc domain, optionally a IgG1 or IgG2A Fc domain.

11. The isolated antibody, or antigen-binding fragment thereof, of any one of claims 1-10, which is covalently attached to a detectable label.

12. The isolated antibody, or antigen-binding fragment thereof, of claim 11, wherein the detectable label is selected from one or more of a fluorophore/fluorescent dye, polymer particle label, metal particle label, iodine-based label, alkaline phosphatase, horseradish peroxidase, luminescent label, radioactive label or radioisotope, nanoparticle, and a quantum dot.

13. A composition, comprising the isolated antibody, or antigen-binding fragment thereof, of any one of claims 1-12, and a suitable carrier.

14. The composition of claim 13, which is lyophilized.

15. A method of determining an amount of a human NRP2 polypeptide in a biological sample, comprising
(a) contacting the biological sample with an antibody, or antigen-binding fragment thereof, of any one of claims 1-12; and
(b) determining the amount of the antibody, or antigen-binding fragment thereof, in the biological sample, which determines the amount of the human NRP2 polypeptide in the biological sample.

16. A method of identifying an NRP2-expressing cancer in a biological sample of cancer tissue from a subject, comprising
(a) contacting the biological sample with an antibody, or antigen-binding fragment thereof, of any one of claims 1-12;
(b) determining the amount or subcellular localization of the antibody, or antigen-binding fragment thereof, in the biological sample, which determines the amount or subcellular localization of NRP2 in the biological sample; and
(c) identifying the NRP2-expressing cancer if (i) the amount of NRP2 in the biological sample of cancer tissue from the subject is increased relative to a control or reference, or (ii) the subcellular localization of the NRP2 is increased relative to a control or reference.

17. The method of claim 16, wherein the subcellular localization of the NRP2 is increased in the nucleus or nuclear envelope relative to the control or reference.

18. The method of claim 16 or 17, wherein (b) comprises determining the ratio of NRP2 localized in the nucleus or nuclear envelope (nuclear NRP2) relative to NRP2 localized on the cell surface (cell surface NRP2), and (c) comprises identifying the NRP2-expressing cancer if the ratio of nuclear NRP2/cell surface NRP2 is increased relative to a control or reference.

19. The method of any one of claims 16-18, comprising administering or causing to be administered to the subject having the NRP2-expressing cancer of (c) at least one NRP2-targeted therapeutic agent, optionally a therapeutic antibody, or antigen-binding fragment thereof, which binds to human NRP2, and optionally in combination with at least one additional anti-cancer therapy or agent.

20. A method of identifying a subject for NRP2-targeted therapy, comprising
(a) contacting a biological sample from the subject with an antibody, or antigen-binding fragment thereof, of any one of claims 1-12;
(b) determining the amount or subcellular localization of the antibody, or antigen-binding fragment thereof, in the biological sample, which determines the amount or subcellular localization of NRP2 in the sample; and
(c) identifying the subject as suitable for NRP2-targeted therapy if (i) the amount of the NRP2 in the biological sample is increased relative to a control or reference, or (ii) the subcellular localization of the NRP2 is increased relative to a control or reference.

21. The method of claim 20, wherein the subcellular localization of the NRP2 is increased in the nucleus or nuclear envelope relative to the control or reference.

22. The method of claim 20 or 21, wherein (b) comprises determining the ratio of NRP2 localized in the nucleus or nuclear envelope (nuclear NRP2) relative to NRP2 localized on the cell surface (cell surface NRP2), and (c) comprises identifying the NRP2-expressing cancer if the ratio of nuclear NRP2/cell surface NRP2 is increased relative to a control or reference.

23. The method of any one of claims 20-22, comprising administering or causing to be administered to the subject of (c) at least one NRP2-targeted therapeutic agent, optionally a therapeutic antibody, or antigen-binding fragment thereof, which binds to human NRP2.

24. A method for identifying a treatment regimen for a subject with castration-resistant prostate cancer (CRPC) that is relapsed/refractory to androgen receptor (AR)-targeted therapy, comprising
(a) contacting a biological sample from the subject with an antibody, or antigen-binding fragment thereof, of any one of claims 1-12;
(b) determining the subcellular localization of the antibody, or antigen-binding fragment thereof, in the biological sample, which determines the subcellular localization of NRP2 in the sample; and
(c) identifying the subject as being suitable for an aggressive treatment regimen if the subcellular localization of the NRP2 in the nucleus or nuclear envelope is increased relative to a control or reference.

25. The method of claim 24, wherein (b) comprises determining the ratio of NRP2 localized in the nucleus or nuclear envelope (nuclear NRP2) relative to NRP2 localized on the cell surface (cell surface NRP2), and (c) comprises identifying the subject as being suitable for the aggressive treatment regimen if the ratio of nuclear NRP2/cell surface NRP2 is increased relative to a control or reference.

26. The method of claim 24 or 25, wherein the aggressive treatment regimen comprises at least one NRP2-targeted therapeutic agent, optionally a therapeutic antibody, or antigen-binding fragment thereof, which binds to human NRP2, in combination with at least one additional anti-cancer therapy or agent optionally at least one chemotherapeutic agent.

27. A method of identifying NRP2-mediated drug resistance in a subject, comprising
(a) contacting a biological sample from the subject with an antibody, or antigen-binding fragment thereof, of any one of claims 1-12;
(b) determining the subcellular localization of the antibody, or antigen-binding fragment thereof, in the biological sample, which determines the subcellular localization of NRP2 in the sample; and
(c) identifying the subject as having NRP2-mediated drug resistance if the subcellular localization of NRP2 to the nucleus or nuclear envelope is increased relative to a control or reference.

28. The method of claim 27, comprising administering or causing to be administered to the subject of (c) at least one NRP2-targeted therapeutic agent, optionally a therapeutic antibody, or antigen-binding fragment thereof, which binds to human NRP2, optionally in combination with at least one additional anti-cancer therapy or agent, optionally radiotherapies, cancer immunotherapies, chemotherapeutic agents (optionally DNA damaging agents, DNA repair inhibitors), hormonal therapeutic agents, kinase inhibitors, anti-growth factor therapies, and androgen receptor (AR)-targeted therapies.

29. The method of any one of claims 26-28, wherein (c) comprises correlating a higher increase in subcellular localization of NRP2 to the nucleus or nuclear envelope with a more advanced stage of NRP2-mediated drug resistance.

30. The method of any one of claims 15-29, comprising first obtaining the biological sample from a subject, optionally by receiving the biological sample from a healthcare provider, optionally wherein the subject has or is suspected of having a cancer.

31. The method of any one of claims 15-30, comprising providing information to a healthcare provider on the amount or subcellular localization of NRP2 in the biological sample.

32. The method of claim 31, wherein the information on the subcellular localization of NRP2 comprises a ratio of nuclear NRP2/cell surface NRP2 in the biological sample.

33. The method of any one of claims 15-32, wherein the biological sample is a biopsy sample.

34. The method of claim 33, wherein the biopsy sample is a cancer or suspected cancer biopsy sample.

35. The method of claim 33 or 34, wherein the biopsy sample is selected from skin tissue, liver tissue, pancreatic tissue, prostate tissue, mesothelial tissue, epithelial tissue, ovarian tissue, colorectal tissue, gastric tissue, brain tissue, lung tissue, kidney tissue, bladder tissue, uterine tissue, esophageal tissue, cervical tissue, testicular tissue, breast tissue, and mesenchymal tissue such as bone tissue, cartilage tissue, fat tissue, muscle tissue, vascular tissue, blood, or hematopoietic cells/tissue, optionally a liquid biopsy.

36. The method of any one of claims 15-35, wherein the control or reference is a reference standard, a biological sample from a healthy subject, or a healthy biological sample from the same subject.

37. The method of claim 36, wherein the control is a non-cancerous biological sample from the same subject, optionally of the same tissue type.

38. The method of any one of claims 15-37, wherein steps (a) and (b) comprise performing an immunohistochemistry (IHC) or immunofluorescence (IF) assay on the biological sample.

39. The method of claim 38, wherein the IHC or IF assay comprises a multiplex IHC or IF assay, comprising contacting the biological sample with at least one additional antibody, or antigen-binding fragment thereof, which specifically binds to an additional marker of interest.

40. The method of claim 39, wherein the additional marker of interest is selected from one or more of signal transduction pathway molecules (VEGF-C, VEGF-A, EGF, IGF, FGF, TGF-beta, VEGFR1, VEGFR2, VEGFR3, CCR7, EGFR1, EGFR2, PDGFR, TGFR1, TGFR2, TGFR3, and c-MET); EMT markers (N-cadherin, E-cadherin, OB-cadherin, ZO-1, α5β1 integrin 1, αVβ6 integrin, Syndecan-1, FSP1, Cytokeratin, α-SMA, Vimentin 1, β-Catenin, CDH1, EPCAM, claudins, cytokeratins, Snail, Slug, ZEB1, ZEB2, and Twist); lymphangiogenesis markers (lymphatic vessel endothelial hyaluronan receptor-1, or LYVE-1); fibrosis markers (collagen fibers, tenascin-C, and α-SMA); and immune activation/exhaustion markers and immune modulators (CD45RA, CD45RO, CD27, CD62L, CD95, PD-1, PD-L1, CD80, CD86, CXCR4, BLC, sCD30, MCP-2, IP-10, APRIL, SIL-2R, IL7, MIF, MIP-1b, SCF, SDF-1a, and sTNF-RI).


SEQUENCE LISTING (27 sequences)

SEQ ID NO: 1 - protein, 10 aa, synthetic construct
  REGION 1..10: note = aNRP2-36v2 VH CDR1
GYSFTDYNMN

SEQ ID NO: 2 - protein, 17 aa, synthetic construct
  REGION 1..17: note = aNRP2-36v2 VH CDR2
VINPKSGTTVYNQKFKG

SEQ ID NO: 3 - protein, 7 aa, synthetic construct
  REGION 1..7: note = aNRP2-36v2 VH CDR3
ADSSGGC

SEQ ID NO: 4 - protein, 16 aa, synthetic construct
  REGION 1..16: note = aNRP2-36v2 VL CDR1
KSTQRLLDSDGKTYLN

SEQ ID NO: 5 - protein, 7 aa, synthetic construct
  REGION 1..7: note = aNRP2-36v2 VL CDR2
LMSKLDS

SEQ ID NO: 6 - protein, 9 aa, synthetic construct
  REGION 1..9: note = aNRP2-36v2 VL CDR3
WQGTHFPWT

SEQ ID NO: 7 - protein, 116 aa, synthetic construct
  REGION 1..116: note = aNRP2-36v2 heavy chain variable region
ELQLQQSGPELVKPGASVKISCKASGYSFTDYNMNWVKQSNGKSLEWIGVINPKSGTTVYNQKFKGKATLTVDQSSSTAYMQLNSLTSEDSAVYYCARADSSGGCWGQGTTLTVSS

SEQ ID NO: 8 - protein, 112 aa, synthetic construct
  REGION 1..112: note = aNRP2-36v2 light chain variable region
DVVMTQTPLTLSVTIGQPASISCKSTQRLLDSDGKTYLNWLLQRPGQSPKRLIYLMSKLDSGVPDRFTGSGSGTDFTLKISRVEAEDLGVYYCWQGTHFPWTFGGGTKLEIK

SEQ ID NO: 9 - protein, 909 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 10 - protein, 904 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 11 - protein, 887 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFADEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 12 - protein, 884 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 13 - protein, 879 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 14 - protein, 833 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLY

SEQ ID NO: 15 - protein, 573 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 16 - protein, 451 aa, Homo sapiens
GSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 17 - protein, 320 aa, Homo sapiens
QCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 18 - protein, 155 aa, Homo sapiens
QCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVT

SEQ ID NO: 19 - protein, 620 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPS

SEQ ID NO: 20 - protein, 589 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSE

SEQ ID NO: 21 - protein, 833 aa, Mus musculus
QQDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSVLYIKFTSDYARQGAGFSLRYEIFKTGSEDCSKNFTSPNGTIESPGFPEKYPHNLDCTFTILAKPRMEIILQFLTFDLEHDPLQVGEGDCKYDWLDIWDGIPHVGPLIGKYCGTKTPSKLRSSTGILSLTFHTDMAVAKDGFSARYYLIHQEPPENFQCNVPLGMESGRIANEQISASSTFSDGRWTPQQSRLHGDDNGWTPNLDSNKEYLQVDLRFLTMLTAIATQGAISRETQKGYYVKSYKLEVSTNGEDWMVYRHGKNHKIFQANNDATEVVLNKLHMPLLTRFIRIRPQTWHLGIALRLELFGCRVTDAPCSNMLGMLSGLIADTQISASSTREYLWSPSAARLVSSRSGWFPRNPQAQPGEEWLQVDLGTPKTVKGVIIQGARGGDSITAVEARAFVRKFKVSYSLNGKDWEYIQDPRTQQTKLFEGNMHYDTPDIRRFDPVPAQYVRVYPERWSPAGIGMRLEVLGCDWTDSKPTVETLGPTVKSEETTTPYPMDEDATECGENCSFEDDKDLQLPSGFNCNFDFPEETCGWVYDHAKWLRSTWISSANPNDRTFPDDKNFLKLQSDGRREGQYGRLISPPVHLPRSPVCMEFQYQAMGGHGVALQVVREASQESKLLWVIREDQGSEWKHGRIILPSYDMEYQIVFEGVIGKGRSGEISIDDIRISTDVPLENCMEPISAFAVDIPETHGGEGYEDEIDDEYEGDWSNSSSSTSGAGDPSSGKEKSWLY

SEQ ID NO: 22 - protein, 832 aa, Rattus norvegicus
QQDPPCGGRLNSKDAGYITSPGYPQDYPSHQNCEWVVYAPEPNQKIVLNFNPHFEIEKHDCKYDFIEIRDGDSESADLLGKHCGNIAPPTIISSGSVLYIKFTSDYARQGAGFSLRYEIFKTGSEDCSKNFTSPNGTIESPGFPEKYPHNLDCTFTILAKPRMEIILQFLTFDLEHDPLQVGEGDCKYDWLDIWDGIPHVGPLIGKYCGTKTPSKLRSSTGILSLTFHTDMAVAKDGFSARYYLVHQEPPENFQCNAPLGMESGRIANEQISASSTFSDGRWTPQQSRLHGDDNGWTPNVDSNKEYLQVDLRFLTMLTAIATQGAISRETQKGYYVKSYKLEVSTNGEDWMVYRHGKNHKVFQANNDATELVLNKLHTPLLTRFIRIRPQTWHLGIALRLELFGCRVTDAPCSNMLGMLSGLIADTQISASSTREYLWSPSAARLVSSRSGWFPRNPQAQPGEEWLQVDLGTPKTVKGVIIQGARGGDSITAMEARAFVRKFKVSYSLNGKDWEYIQDPRTQQPKLFEGNMHYDTPDIRRFEPVPAQYVRVYPERWSPAGIGMRLEVLGCDWTDSKPTVETLGPTVKSEETTTPYPMDEDATECGENCSFEDDKDLQLPSGFNCNFDFPEETCGWMYDRAKWLQSTWISSANPNDRTFPDDKNFLKLQSDGGREGQFGRLISPPVHLPRSPVCMEFQYQAMGGHGVALQVVREARQESKLLWVIREDQGSEWKHGRIILPSYDMEYQIVFEGVIGKGRSGEISIDDIRISTDVPLENCMEPISAFAVDIPEIHGGEGYEDEIDDDYEGDWNNSSSTSGAGSPSSGKEKSWLY

SEQ ID NO: 23 - protein, 832 aa, Macaca mulatta
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIVLQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTHEYLWSPSAARLVSSRAGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPVPAQYVRVYPERWSPAGIGMRLEVLGCDWTDSKPTVETLGPTVKSEETTTPYPTDEEATECGENCSFEDDKDLQLPSGFNCNFDFPEEPCGWMYDHAKWLRTTWASSSSPNDRTFPDDRNFLRLQSDSRREGQYARLISPPVHLPQSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSATSGSGAPSTDKEKSWLY

SEQ ID NO: 24 - protein, 581 aa, Homo sapiens
FRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDE

SEQ ID NO: 25 - protein, 18 aa, Homo sapiens
SGFNCNFDFLEEPCGWMY

SEQ ID NO: 26 - protein, 12 aa, Homo sapiens
VDIPEIHEREGY

SEQ ID NO: 27 - protein, 810 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYW